(19) United States
(12) Patent Application Publication
(10) Pub. No.: US 2019/0365720 A1
(43) Pub. Date: Dec. 5, 2019

(54) Novel formulations of amidine substituted beta-lactam compounds on the basis of modified cyclodextrins and acidifying agents, their preparation and use as antimicrobial pharmaceutical compositions

(71) Applicant: AiCuris Anti-Infective Cures GmbH, Wuppertal (DE)

(72) Inventors: Yogeshwar BACHHAV, Mumbai (IN); Susanne BONSMANN, Koln (DE); Tamara PFAFF, Dusseldorf (DE)

(73) Assignee: AiCuris Anti-Infective Cures GmbH, Wuppertal (DE)

(21) Appl. No.: 16/461,865

(22) PCT Filed: Nov. 17, 2017
(86) PCT No.: PCT/EP2017/079638

§ 371 Date: May 17, 2019

(30) Foreign Application Priority Data
Nov. 18, 2016 (EP) 16199662.4

(51) Int. Cl. A61K 31/427; A61K 47/12; A61K 47/40; A61K 9/19; A61K 9/08; A61P 31/04; A61K 31/4439

(52) U.S. Cl. CPC A61K 31/427; A61K 47/12; A61K 47/40; A61K 9/0019; A61K 9/08; A61P 31/04; A61K 31/4439; A61K 9/19

(57) ABSTRACT

The present invention discloses novel formulations, reconstitutable solid compositions, pharmaceutical compositions, and aqueous injectable formulations of specific amidine substituted beta-lactam compounds on the basis of modified cyclodextrins and organic and/or inorganic acids, their preparation and use as antimicrobial pharmaceutical compositions that are parenterally or orally administrable.

### FIELD OF THE INVENTION

The present invention relates to novel formulations of amidine substituted β-lactam compounds on the basis of modified cyclodextrins and acidifying agents, their preparation and use as antimicrobial pharmaceutical compositions. Specifically, by the provided formulations of the present invention said amidine substituted β-lactam compounds are parenterally, preferably intravenously (hereinafter i.v.), and orally administrable.

In particular, this invention relates to novel formulations of specific β-lactam compounds which are synthetic amidine substituted monobactam derivatives with a sulphobutylether-β-cyclodextrin (hereinafter SBE-β-CD) and further with specific organic and/or inorganic acids useful as antimicrobial agents that are to be administered parenterally, preferably i.v., or orally and their preparation.

### BACKGROUND OF THE INVENTION

The emergence and spread of antibiotic resistant bacteria is one of the major public health problems of the current century. Specifically, the spread of antibiotic resistant bacteria has reached an unprecedented dimension. While the most resistant isolates continue to emerge in the hospital setting, physicians and epidemiologists are encountering increasing numbers of resistant bacteria in the community among people without previous healthcare contact. The number of patients who are dying from untreatable nosocomial infections continues to grow. Therapeutic options are especially limited for infections due to multi-drug-resistant Gram-negative pathogens including Enterobacteriaceae and non-fermenters, a situation made even worse by the fact that the pipelines of the pharmaceutical industry contain few compounds with promising resistance breaking profiles. Thus, there is a need to increase the number of effective antimicrobial drugs to defeat infections caused by bacteria that have become resistant to existing medicines (Jim O'Neill; The Review on Antimicrobial Resistance; Tackling drug-resistant infections globally: Final Report and Recommendations; May 2016).

### β-Lactam Compounds

The highly successful and well-tolerated class of β-lactam antibiotics has historically been one mainstay for the treatment of infections caused by Gram-negative pathogens. Among these especially third generation cephalosporins, carbapenems and monobactams are extensively used for the treatment of infections with Gram-negative bacteria. However, a vast array of more than 1000 β-lactamases and further resistance mechanisms severely endanger the mid-term usability of the current compounds in these subclasses. Especially extended-spectrum β-lactamases (ESBLs) and carbapenemases are important drivers of resistance.

New β-lactams with effective resistance breaking properties and respective pharmaceutical formulations thereof, so to make them clinically administrable to a patient in need of antimicrobial therapy, are urgently needed to fill the gap.

WO 2013/110643 A1 describes amidine substituted monobactam derivatives of the general formula:

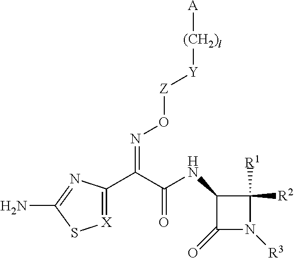

in which
R¹ and R² independently of one another represent hydrogen, aminocarbonyl or (C₁-C₄)-alkyl, or
R¹ and R² together with the carbon atom to which they are bonded form a (C₃-C₈)-cycloalkyl,
R³ represents —(CH₂)ₘ—(SO₂)OH or —O—(CH₂)ₒ—(SO₂)OH,
   wherein m and o independently of one another represent an integer 0, 1, 2 or 3, and
   wherein any CH₂-group contained in the residues which R³ represents may be substituted with one or two (C₁-C₄)-alkyl-residues,
X represents CR⁴ or N,
R⁴ represents hydrogen or halogen,
Z represents a bond or an alkyl-chain having one, two, three or four carbon atoms,
   whereby the alkyl-chain may be substituted with one, two, three or four substituents, selected independently of one another from the group consisting of carboxy, aminocarbonyl and (C₁-C₄)-alkyl,
   whereby alkyl in turn may be substituted with a substituent selected from the group consisting of hydroxy, carboxy and aminocarbonyl,
Y represents a bond, O, NH or S,
A represents (C₆-C₁₀)-aryl or 5- to 10-membered heteroaryl,
   whereby aryl and heteroaryl are substituted with a substituent of the following formula

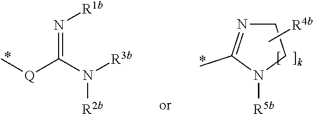

wherein
R^{1b}, R^{2b} and R^{3b} independently of one another represent hydrogen, amino, hydroxy, (C₁-C₄)-alkyl, (C₁-C₄)-alkoxy, (C₃-C₆)-cycloalkyl, 4-, 5-, 6- or 7-membered heterocyclyl or 5- or 6-membered heteroaryl,
whereby amino and hydroxy may be substituted with one or two substituents selected independently of one another from the group consisting of carbonyl, (C₁-C₄)-alkylcarbonyl, mono- or di-(C₁-C₄)-alkylaminocarbonyl, and (C₁-C₄)-alkyl,
whereby alkoxy, heterocyclyl and heteroaryl may be substituted with one, two or three substituents selected independently of one another from the group consisting of halogen, hydroxy, amino, carbonyl, carboxy, (C₁-C₄)-alkylcarbonyl, (C₁-C₄)-alkoxy, mono- or di-(C₁-C₄)-alkylamino, mono- or di-(C₁-C₄)-alkylaminocarbonyl, NH—CH(═NH), —NH—C(═NH)(NH₂), C(═NH)CH₃ and (C₁-C₄)-alkyl, and
whereby alkyl and cycloalkyl may be substituted with one, two or three substituents selected independently of one another from the group consisting of halogen, hydroxy, amino, carbonyl, carboxy, carbonyloxy, aminocarbonyl, carbonylamino, (C₁-C₄)-alkylcarbonyl, (C₁-C₄)-alkoxy, mono- or di-(C₁-C₄)-alkylamino, mono- or di-(C₁-C₄)-alkylaminocarbonyl, —NH—CH(═NH), —NH—C(═NH)(NH₂), —CH(═NH)CH₃, (C₆-C₁₀)-aryl, 5- or 6-membered heteroaryl and 5- or 6-membered heterocyclyl,
whereby heteroaryl and heterocyclyl in turn may be substituted with (C₁-C₄)-alkyl,
whereby amino in turn may be substituted with 5- or 6-membered heteroaryl, or
R^{2b} and R^{3b} together with the nitrogen atom to which they are bonded form a 5- to 7-membered heterocycle including one, two or three further heteroatoms selected from the series N, O and S and R^{lb} is as defined above,
R^{4b} represents hydrogen, amino, hydroxy, (C₁-C₄)-alkyl or (C₁-C₄)-alkoxy,
   whereby amino and hydroxy may be substituted with one or two substituents selected independently of one another from the group consisting of (C₁-C₄)-alkylcarbonyl, mono- or di-(C₁-C₄)-alkylaminocarbonyl and (C₁-C₄)-alkyl,
   whereby alkoxy may be substituted with one, two or three substituents selected independently of one another from the group consisting of halogen, hydroxy, amino, carbonyl, carboxy, (C₁-C₄)-alkylcarbonyl, (C₁-C₄)-alkoxy, mono- or di-(C₁-C₄)-alkylamino, mono- or di-(C₁-C₄)-alkylaminocarbonyl, —NH—CH(═NH), —NH—C(═NH)(NH₂), —CH(═NH)CH₃ and (C₁-C₄)-alkyl, and
   whereby alkyl may be substituted with one, two or three substituents selected independently of one another from the group consisting of halogen, hydroxy, amino, carbonyl, carboxy, aminocarbonyl, (C₁-C₄)-alkylcarbonyl, (C₁-C₄)-alkoxy, mono- or di-(C₁-C₄)-alkylamino, mono- or di-(C₁-C₄)-alkylamino-carbonyl, —NH—CH(═NH), —NH—C(═NH)(NH₂), —CH(═NH)CH₃, (C₁-C₄)-alkyl, (C₆-C₁₀)-aryl and 5- or 6-membered heteroaryl,
R^{5b} represents hydrogen or (C₁-C₄)-alkyl,
Q represents a bond, CH₂ or NH,
k represents an integer 1 or 2, and
* is the linkage site to the residue represented by A, and
   whereby aryl and heteroaryl further may be substituted with one or two substituents selected independently of one another from the group consisting of halogen, cyano, amino, hydroxy, (C₁-C₄)-alkyl, (C₁-C₄)-alkoxy, mono- or di-(C₁-C₄)-alkylamino, amino-(C₁-C₄)-alkyl, hydroxy-(C₁-C₄)-alkyl or carboxy,
   whereby alkyl, alkoxy, alkylamino, aminoalkyl, hydroxyalkyl and carboxy in turn may be substituted with a substituent selected from the group consisting of halogen, (C₁-C₄)-alkyl and carbonyl, and
l represents an integer 0, 1, 2 or 3,
   and the salts thereof, the solvates thereof and the solvates of the salts thereof.

In view of the increasing resistance development of pathogenic bacteria against known antimicrobial and antibacterial agents, including multiple resistances, the ongoing need to find novel antibacterial substances has been addressed by the above outlined compounds with different structural motives.

WO 2013/110643 A1 also describes cyclodextrin-free pharmaceutical formulations of the compounds mentioned therein.

### Problems Underlying the Invention

The specific amidine substituted monobactam derivative compounds according to the formulae (I)-(VII):

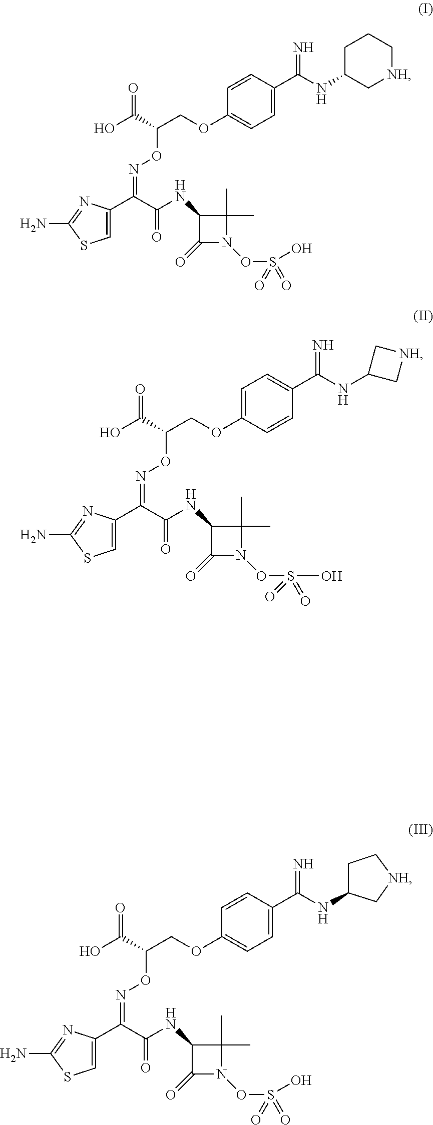 [SMILES: CC1(C)[C@H](NC(=O)/C(=N\O[C@@H](COc2ccc(C(=N)N[C@@H]3CCCNC3)cc2)C(=O)O)c2csc(N)n2)C(=O)N1OS(=O)(=O)O ; CC1(C)[C@H](NC(=O)/C(=N\O[C@@H](COc2ccc(C(=N)NC3CNC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCNC3)cc2)C(=O)O)c2csc(N)n2)C(=O)N1OS(=O)(=O)O]

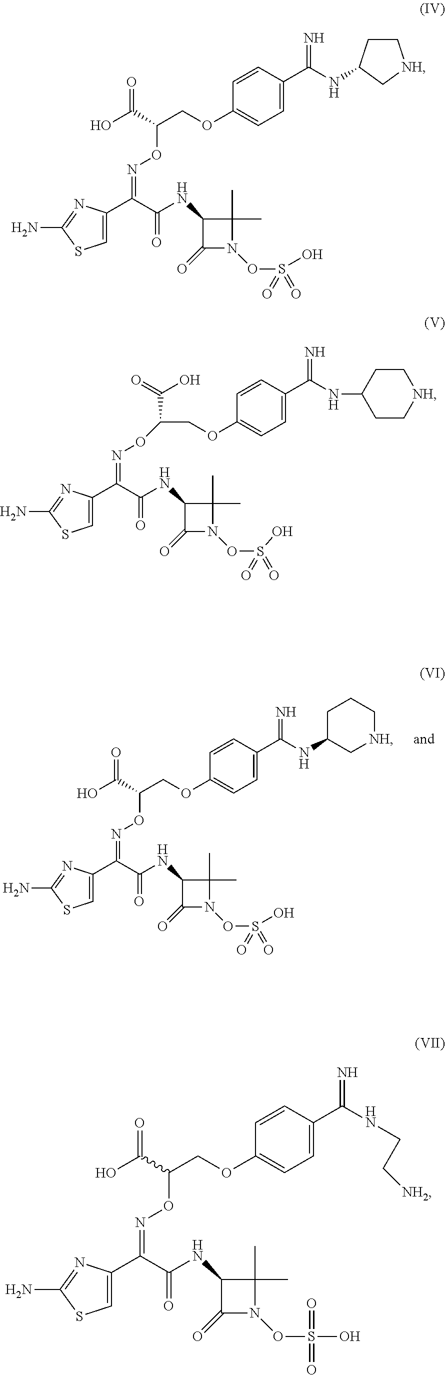 [SMILES: CC1(C)[C@H](NC(=O)/C(=N\O[C@@H](COc2ccc(C(=N)N[C@@H]3CCNC3)cc2)C(=O)O)c2csc(N)n2)C(=O)N1OS(=O)(=O)O ; CC1(C)[C@H](NC(=O)/C(=N\O[C@@H](COc2ccc(C(=N)NC3CCNCC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCCNC3)cc2)C(=O)O)c2csc(N)n2)C(=O)N1OS(=O)(=O)O ; "and" ; CC1(C)[C@H](NC(=O)/C(=N\OC(COc2ccc(C(=N)NCCN)cc2)C(=O)O)c2csc(N)n2)C(=O)N1OS(=O)(=O)O]

and the salts thereof, the solvates thereof and the solvates of the salts thereof, are resistance-breaking β-lactam antibiotics (see WO 2013/110643 A1; which is hereby incorporated by reference) and represent the active pharmaceutical ingredients (hereinafter API(s)) of the formulations, reconstitutable solid compositions, pharmaceutical formulations and aqueous injectable solutions of invention.

These APIs in accordance with the invention possess, however, a zwitterionic character. Thus, these compounds are ionizable molecules with several different pKa values. Accordingly, the solubility and stability of these compounds is highly pH-dependent (see FIG. 23) which poses significant problems and challenges for formulating either parenteral administration forms thereof or oral administration forms, respectively.

For instance, said APIs of the formulae (I)-(VII) are very unstable at basic pH ranges and are per se not stable in dissolved state; e.g. when dissolved in water.

Thus, the finding of an aqueous, parenterally administrable formulation of the compounds according to the formulae (I)-(VII) with a sufficient shelf life for clinical use is challenging. These problems are magnified by the above-mentioned zwitterionic nature of the APIs which means that these compounds are not readily solubilised by conventional excipients such as oils, surfactants or water miscible co-solvents and through usage of sugars and polymers etc.

Hence, for the APIs of the formulae (I)-(VII), although having good water-solubility at lower pH ranges (see FIG. 23), upon static dilution in physiological pH ranges around pH 7.4, as with parenteral administration forms, rapid precipitation of these compounds has been observed previously. Though suitable pH ranges might be not that challenging for oral dosage forms of the compounds of the formulae (I)-(VII), its zwitterionic character still provided for hurdles that could not have been overcome by conventional formulations with the above stated excipients.

Following this, parenterally and/or orally administrable formulations for clinical use are required which provide for suitable solubility of the APIs of the formulae (I)-(VII) in physiological pH ranges as well as for sufficient stability thereof, whereby precipitation is prevented. Thereby, inter alia the formulations must be provided at an acceptable pH for injection, i.e. a pH 4.0-pH 8.0, and they must be stable for at least 4 to 5 hours storage at ambient temperature, so to allow for administration in a clinical setting.

Specifically, upon i.v. injection, the pH of a drug solution at the site of injection will almost instantaneously increase from the pH of the solution to a pH of approx. 7.4 on a physiological basis. The solubility of the compounds mentioned above at pH 7.4 is, however, up to five times lower under such pH (see FIG. 23). Thus, precipitation of these compounds at the injection site is likely. It should be mentioned that drug precipitation after i.v. administration, though very common, is undesirable for a medicinal product. Hence, such formulations are not clinically administrable.

Therefore, it is an object of the invention to solubilize the APIs of the formulae (I)-(VII), preferably of formula (I), in a stable, storable and clinical ready-to-use formulation that is administrable parenterally and/or orally. Thereby, precipitation of said APIs shall be prevented upon dilution in aqueous media in case of parenteral administration. Moreover, it is an object of the invention to provide for such clinical ready-to-use formulations of the APIs which are also stable upon storage.

Thus, it is also an object of the invention to provide for clear aqueous injectable solutions of the compounds (I)-(VII) with a pH 4.0 and required drug loading thereof. Simultaneously, specifically for i.v. injection of these formulations, an in-use stability for at least 6 hours up to 24 hours at room temperature is desirous.

In sum, the amidine substituted monobactam derivative compounds of the formulae (I)-(VII) and the salts thereof, the solvates thereof and the solvates of the salts thereof as APIs are challenging to formulate
a) in an aqueous parenteral formulation that is sufficiently concentrated with API (i.e. sufficient drug load) and stable, and present in a medium having a physiologically acceptable pH for parenteral, particularly i.v. administration;
b) in oral administration forms such as capsules and tablets, whereby also the API is present with sufficient drug load and stable, though pH constraints are more lax for oral route, as the person skilled in the art is aware of.

### Cyclodextrins and Modified Derivatives Thereof

Cyclodextrins are known for their use in increasing solubility of drugs by forming inclusion complexes with hydrophobic molecules. Cyclodextrins are cyclic carbohydrates derived from starch. One has to differ among “unmodified cyclodextrins” and “modified cyclodextrins”:

The “unmodified cyclodextrins” differ by the number of glucopyranose units joined together in the cylindrical structure. The parent cyclodextrins contain 6, 7, or 8 glucopyranose units and are referred to as α-, β-, and γ-cyclodextrin respectively.

Each cyclodextrin subunit has secondary hydroxyl groups at the 2- and 3-positions and a primary hydroxyl group at the 6-position. The cyclodextrins may be pictured as hollow truncated cones with hydrophilic exterior surfaces and hydrophobic interior cavities. In aqueous solutions, these hydrophobic cavities provide a haven for hydrophobic organic compounds, which can fit all, or part of their structure into these cavities. This process, known as “inclusion complexation”, may result in increased aqueous solubility and stability for the complexed drug. The complex is stabilized by hydrophobic interactions and does not involve the formation of any covalent bonds.

Chemical modification of the parent cyclodextrins (usually at the hydroxyl moieties) has resulted in “modified cyclodextrins” which are “cyclodextrin derivatives” with sometimes improved safety, stability and solubility while retaining or improving the complexation ability of the cyclodextrin itself. Of the numerous derivatized cyclodextrins prepared to date, only two appear to be commercially relevant, namely the 2-hydroxypropyl derivatives (HP-β3-CD), neutral molecules being commercially developed by Janssen and others, and the sulfoalkyl ether derivatives (SAE-f3-CD), being developed by CyDex, Inc.

Naturally-occurring cyclodextrins such as beta cyclcodextrins are used in a variety of pharmaceutical applications. However natural cyclodextrins show very low solubility and its also linked to nephrotoxicity. Availability of multiple reactive hydroxyl groups, the functionality of native cyclodextrisn provides an opportunity to come up with the modfifed cyclodextrins. Examples for the term modified in the context of “modified cyclodextrins” are unsubstituted or native cyclodextrins that have been chemically modified in order to improve their properties. These derivatives are mainly based on hydroxyalkylation or alkylation or sulfoalkylation of the (C-2, C-3 or C-6 hydroxyls and the principal aim of these substitutions is to improve the solubility of the natural product. There is an infinite number of possible derivatives of cyclodextrins. The most important chemically modified cyclodextrins, which may be used in context of the present invention are hydroxypropyl betacyclodextrin (HPBCD), randomly methylated betacyclodextrin (RAMEB), heptakis(2,6-dimethyl)-betacyclodextrin (DIMEB). Another modified cyclodextrin is Captisol, which is a polyanionic beta-cyclodextrin derivative with a sodium sulfonate salt separated from the lipophilic cavity by a butyl ether spacer group, or sulfobutylether (SBE).

### SBE-β-CD (Captisol®)

The sulfoalkyl ether derivatives (SAE-β-CD) represent a class of negatively charged cyclodextrins, which vary in the nature of the alkyl spacer, the salt form, the degree of substitution and the starting parent cyclodextrin. The sodium salt of the sulfobutyl ether derivative of β-cyclodextrin (SBE-β-CD), with an average of about 7 substituents per cyclodextrin molecule, is being commercialized by CyDex, Inc. (Kansas) together with Ligand Pharmaceuticals, Inc., as Captisol® cyclodextrin.

β-cyclodextrin (β-CD) and other cyclodextrins (CDs) already have utility for solubilizing and stabilizing drugs; however, some are nephrotoxic when administered parenterally or lead to precipitation of the drugs upon i.v. administration due to its low aqueous solubility. It has been attempted to identify, prepare, and evaluate various cyclodextrin derivatives with superior inclusion complexation and maximal in vivo safety for various biomedical uses. A systematic study led to said SBE-β-CD (i.e. Captisol®), a polyanionic variably substituted sulfobutyl ether of 3-CD, as a non-nephrotoxic derivative and HP-β-CD, a modified CD developed by Janssen.

SBE-β-CD alone has undergone extensive safety studies and is inter alia currently used in six products approved by the Food and Drug Administration (FDA).

With HP-β-CD there are quite more formulations on the market, and HP-β-CD is also already used for oral route medications.

Technical Problems with Cyclodextrins and Modified Cyclodextrins

Moreover, it has been suspected that underivatised or unmetabolised pure cyclodextrin has toxic effects on the body and so is unsuitable as a pharmaceutical excipient, particularly when administered parenterally (see e.g. Cyclodextrins. Stella V J. Toxicol Pathol. 2008 January; 36(1):30-42).

Aside from unwanted side effects, additional problems are associated with parenteral administration of a drug in a surfactant-based vehicle.

For instance, by virtue of their respective functional groups, derivatized cyclodextrins can differ in terms of their state of ionization when present in solutions at different pH values. The functional group of carboxy-β-cyclodextrins, e.g. succinyl-β-cyclodextrin, typically has a pKa of approximately 3 to 5.

Thus, carboxy cyclodextrins typically are charged in solutions at pH 3.5 to 14. As the pH decreases below the pKa of the functional groups of carboxy-β-cyclodextrin, the overall negative charge of the cyclodextrin decreases. The ionization state for neutral cyclodextrins such as HP-3-CD does not change over the pharmaceutically relevant pH range. However, the sulfoalkyl ether cyclodextrins (SAE-β-CDs), unlike most cyclodextrins, has a pKa of less than 1, meaning that in solution, the SAE-f3-CD remains fully ionized throughout the pH-range usable for drug formulation (i.e. pH 1 to 14). Although no literature is available regarding the change in ionization versus solution pH for the sulfate derivatized cyclodextrin, it is assumed that the sulfate derivatized cyclodextrins are also fully ionized over the pH range of 1 to 14. Unfortunately, there are many drugs for which cyclodextrin complexation either is not possible or produces no apparent advantages as disclosed by e.g. J. Szejtli, Cyclodextrins in Drug Formulations: Part II, Pharmaceutical Technology, 24-38, August, 1991.

Nevertheless, cyclodextrins and their derivatives are widely used either in liquid formulations to enhance the aqueous solubility of hydrophobic compounds, or in oral formulations for achieving the same effects.

Yet it is known that SBE-β-CD interacts very well with neutral drugs to facilitate solubility and chemical stability, and because of its polyanionic nature, it interacts particularly well with cationic drugs [LIT.].

However, the drugs to be formulated by the present invention (i.e. the compounds (I) to (VII) as API) have zwitterionic character and are thus strongly pH-dependent in terms of solubility and stability, as well as very hydrophilic with negative log P values.

All in all, a need remains for improved formulations that are readily dilutable from a concentrated solution while maintaining clarity of the API in dissolved state, which can be thus administered parenterally at a physiologically acceptable pH, and which remain chemically stable under a variety of storage conditions, and which are easy to handle and to administer.

### BACKGROUND ART

In this regard, the following background art for the subject matter of the present invention is briefly summarized:

U.S. Pat. No. 6,267,985 to Chen et al. discloses a method for improving the solubilization of triglycerides and improved delivery of therapeutic agents. The disclosed formulations comprise a combination of two surfactants, a triglyceride and therapeutic agent that is capable of being solubilized in the triglyceride, the carrier, or both the triglyceride and the carrier. The '985 patent suggests the use of aniodarone and of an optional solubilizing agent, such as a cyclodextrin, which can include cyclodextrin derivatives such as hydroxypropyl cyclodextrin (HP-β-CD), sulfobutyl ether cyclodextrin and a conjugate of sulfobutyl ether cyclodextrin. HP-β-CD is the preferred cyclodextrin.

U.S. Pat. No. 6,294,192 to Patel et al. discloses triglyceride-free oral pharmaceutical compositions capable of solubilizing therapeutically effective amounts of hydrophobic therapeutic agents. The disclosed formulations include a combination of a hydrophilic surfactant and a hydrophobic surfactant. The '192 patent suggests the use of amiodarone and of an optional solubilizing agent, such as a cyclodextrin, which can include cyclodextrin derivatives such as HP-β-CD and sulfobutyl ether cyclodextrin. HP-β-CD is the preferred cyclodextrin.

U.S. patent application Ser. No. 20020012680 to Patel et al. discloses triglyceride-free pharmaceutical compositions comprising a hydrophobic therapeutic agent, and a carrier comprising at least one hydrophilic surfactant and at least one hydrophobic surfactant. The application claims but does not teach the use of amiodarone as a suitable hydrophobic therapeutic agent. The claimed formulation can further comprise a solubilizer, which may be a sulfobutyl ether cyclodextrin.

U.S. Pat. Nos. 5,874,418 and 6,046,177 to Stella el al. disclose sulfoalkyl ether cyclodextrin-containing solid pharmaceutical compositions and formulations, and methods for their preparation for the sustained, delayed or controlled delivery of therapeutic agents. The patents disclose formulations containing a physical mixture of a sulfoalkyl ether cyclodextrin and a therapeutic agent, and optionally at least one release rate modifier. Both patents teach that the relative increase in the solubility of a poorly soluble drug in the presence of sulfoalkyl ether cyclodextrins (SAE-β-CDs) is a product of the binding constant and the molar concentration of SAE-β-CD present. Amiodarone is listed as one of a large number of drugs that can be used.

U.S. Pat. Nos. 5,134,127 and 5,376,645 to Stella et al. disclose parenteral formulations containing an SAE-β-CD and a drug. Moreover, Captisol®-based technologies for formulations are e.g. known from VFend®, which is the lyophilized formulation of Voriconazole. Additional FDA approved drugs containing Captisol® include Nexterone®, Geodon®, Abilify®, Naxofil®.

U.S. Pat. No. 6,632,803 B1 describes Voriconazole Captisol® formulations. US 2004/0077594 A1 describes Aripiprazole (Ablilify) Captisol® formulations. US 20030216353 A1 describes Nexterone (Aminodarone) Captisol® formulations. WO2012/005973 A1 describes Noxafil (Posacondazol) Captisol® formulations.

SBE-β-CD and HP-β-CD are also in use in numerous clinical and preclinical studies.

By forming inclusion complexes with cyclodextrins and modified cyclodextrins, major changes in drug candidate properties, including enhanced solubility, physical and chemical stability, and other physicochemical properties, have been well documented (e.g. in Szejtli, 1988; Duchene, 1987; Frömming and Szejtli, 1994; Uekama et al., 1994; Albers and Muller, 1995; Loftsson, 1995; Loftsson and Brewster, 1996; Rajewski and Stella, 1996; Irie and Uekama, 1997; Stella and Rajewski, 1997; Thompson, 1997; Stella et al., 1999; Szente and Szejtli, 1999; Mosher and Thompson 2002; Stefdnsson and Loftsson, 2003; Rao and Stella, 2003; Challa et al., 2005).

These changes have then resulted in better biological performance, e.g. higher bioavailability, and thus, in the use of cyclodextrins and modified cyclodextrins in various commercially successful pharmaceutical products.

By far the greatest advantage has been in the area of enhanced solubility of problematic drugs, predominantly hydrophobic drugs. The use of cyclodextrins to increase physical and chemical stability of drugs in solution and in other dosage forms has been well documented in the literature as well (see e.g. Loftsson and Brewster, 1996).

Generally, cyclodextrins can enhance the stability or catalyze the degradation of some drug molecules, although there are more examples of the latter than the former.

However, the specific nature of their interaction is also a weakness in that only molecules with the right size, geometry, and intrinsic solubility properties benefit from their use. Specifically, hydrophobic drugs with poor intrinsic solubility profiles are known to benefit from cyclodextrin and modified cyclodextrin inclusion complexes in terms of pharmaceutical formulation development.

Thus, from the outset, binding very hydrophilic substances in the hydrophobic inner inclusion complexes with cyclodextrins or modifications thereof, appear to be not a useful measure when aiming at pharmaceutical formulations of such substances for parenteral or oral administration.

### Solution by the Invention

With the given background above, surprisingly and unexpectedly, the present inventors have found that the specific amidine substituted monobactam derivative compounds according to the aforementioned formulae (I) to (VII)—with zwitterionic character and being hydrophilic in nature (i.e. with negative log P value)—by admixture of specific modified cyclodextrins in combination with specific organic and/or inorganic acids as recited herein, can be dissolved in parenterally and/or orally administrable formulations with desirous solubitity and stability properties.

This is even more suprising since, mainly, such modified cyclodextrins are used to solubilize hydrophobic (i.e. lipophilic) compounds/drugs with positive log P values.

The inventors further found that the aqueous formulations of the invention can be provided as a reconstitutable solid composition by e.g. lyophilisation, and upon reconstitution in suitable aqueous media such as Ringer's lactate solution, can be provided as an aqueous injectable solution for clinical parenteral administration.

Specifically, sulphobutyl ether betacyclodextrin (SBE-β-CD) was found by the inventors to significantly increase the water solubility of the compounds (I) to (VII) in formulation, preferably of compound (I), particularly when used at concentrations of 20% w/v. When combined with an acidifying agent such as citric acid (hereinafter CA), solubility was even further enhanced and API precipitation can be prevented.

Particularly, the inventors found solutions with a concentration of 32 mg/mL of API according to the aforementioned formulae (I) to (VII), containing SBE-β-CD with 20% w/v and 1% w/v CA were also physically stable for at least 24 hours at room temperature, provided that no pH adjustment was performed prior to lyophilization. Following lyophilisation, the lyophilisate is reconstitutable with a suitable medium to obtain a final reconstituted solution with pH values between 4.0 and 4.5 and an osmolality of 290-400 mOsm/Kg.

Thereby, the inventors found that, specifically, SBE-β-CD in combination with CA surprisingly supports a higher drug loading of 1% to 15% in the dry state (i.e. a lyophilisate). However, sulphobutyl ether betacyclodextrin or CA alone in formulation is not able to provide the desired drug loading and stability as that of the combination.

The inventors further found that when prepared at larger scale (e.g. at 65 L) and in repeated experiments, the formulations of the invention were robust and can be successfully lyophilised in 50 mL vials and placed under various ICH storage conditions. For instance, after 12 months storage at 25° C./60% relative humidity (RH) and 2-8° C. these formulations were found to be stable. In addition the reconstitution of the lyophilisate shows an in use stability of 6 h at RT using Ringer's lactate buffer solution.

### Advantages of the Present Invention

Thus, the specific combinations of modified cyclodextrins and organic and/or inorganic acids in the formulations of the invention as recited herein, resulted in higher solubilization and improved shelf life of the APIs of the formulae (I) to (VII), and further prevented the occurence of any precipitation during i.v. injection or through an i.v. drip tube.

Specifically, the combinations of SBE-β-CD and CA in the formulations of the invention resulted in higher solubilization and improved shelf life of the APIs of the formulae (I) to (VII), and further prevented the occurence of any precipitation during i.v. injection or through an i.v. drip tube.

Surprisingly, by the formulations of the invention the APIs of the formulae (I) to (VII) are also well absorbed orally with a sufficient bioavailability of >70%, preferably >80%, more preferably >90%, e.g. allowing patients to be switched between intravenous and oral administration of the formulations of the invention. Thus, it is another advantage of the invention that the complexes of APIs with modified cyclodextrins in the dry solid compositions of the invention (e.g. as a lyophilisate) may also be compressed into a tablet or may be filled into capsules.

The modified cyclodextrins in the ranges of the invention do not affect appearance or pH of solution but protects API of the formulae (I) to (VII) from degradation, whereby simultaneously high CA amounts would have negative influence on stability of said APIs.

The API-complexes of the invention with modified cyclodextrins in combination with specific organic and/or inorganic acids have been shown to rapidly dissociate after parenteral drug administration, to have no tissue-irritating effects e.g. after intramuscular dosing, and to result in superior oral bioavailability of poorly water-soluble drugs.

### DESCRIPTION OF THE INVENTION

### Intravenous Administration Route

As outlined above, the APIs of the invention according to the aforementioned formulae (I) to (VII), and the salts thereof, the solvates thereof and the solvates of the salts thereof, are zwitterionic/hydrophilic in nature and thus have poor water solubility and stability in physiologic pH ranges, which makes them difficult to readily formulate as an aqueous pharmaceutically injectable formulation and/or in suitable oral dosage forms such as tablets or capsules.

In accordance with the present invention, it has now been found by the inventors that the water-solubility and stability of the compounds of the aforementioned formulae (I) to (VII) and the salts thereof, the solvates thereof and the solvates of the salts thereof may be sufficiently increased to allow it to be formulated as an aqueous injectable solution by complexing these compounds with a modified cyclodextrin, particularly with SBE-β-CD, in combination with an organic and/or an inorganic acid.

In effect, the modified cyclodextrin inhibits precipitation of the API but keeps them also solubilized and stable at the site of injection. The aqueous injectable formulation containing the complex of API of the aforementioned formulae (I) to (VII) and the modified cyclodextrin, and further an organic and/or an inorganic acid, may be thus administered parenterally, preferably intravenously.

Specifically, substituted β-cyclodextrins were found to significantly increase the water solubility and stability of the APIs of the invention, particularly when used at concentrations of 20% w/v. When combined with an acidifying agent, such as CA, solubility was even further enhanced and the tendency for API precipitation was simultaneously decreased (upon storage as lyophilizate).

Thus, the present invention seeks to overcome some or all of the disadvantages inherent in known parenteral formulations of the APIs of the aforementioned fonnulae (I) to (VII). The invention provides a modified cyclodextrin-based parenteral formulation of specific amidine substituted beta-lactam compounds in combination of organic and/or inorganic acids. The invention provides a clinically viable formulation that can be prepared and stored as dry solid composition (i.e. e.g. a lyophilisate) at a wide range of physiologically acceptable pH values and concentrations of API without significant precipitation of the amidine substituted beta-lactam compounds in vitro and in vivo. The formulation is pharmaceutically stable with a wide range of buffers, saline, or lactated Ringer's solutions.

The formulations of the invention can be prepared as a clear aqueous solution at pH 4.0-4.5 suitable for i.v. administration that is sterilized by sterile filtration and other conventional methods. The liquid formulation is stable under a variety of storage conditions and can also be converted to a reconstitutable solid; e.g. a lyophilisate (freeze dried).

Thus, the formulations of the invention may be formed of dry physical mixtures/complexes of API according to the compounds (I) to (VII) and the modified cyclodextrins, or dry inclusion complexes thereof, which upon addition of a suitable medium, e.g. water for injection (WFI) or Ringer's lactate solution, are reconstituted and may be further diluted to form an aqueous injectable formulation.

Alternatively, within the context of the instant invention, the aqueous injectable formulations may be freeze-dried and later reconstituted with WFI or Ringer's lactate solution and suitable i.v. injectable diluent. Thus, the inclusion complexes in accordance with the invention, may be pre-formed, formed in situ or formed in vivo. All of the above are contemplated by the present invention.

The pharmaceutical formulations of the invention can be administered by injection at a physiologically acceptable pH range.

Accordingly, one main aspect of the invention provides for a clear liquid formulation with pH 4.0-4.5 comprising at least a therapeutically effective amount of an API in accordance with the formulae (I)-(VII), and a modified cyclodextrin, particularly SBE-β-CD, and an organic and/or an inorganic acid, particularly CA, present in an amount sufficient to provide a clear solution at pH 4.0 and to avoid precipitation when diluted with a pharmaceutically acceptable liquid excipient composition.

The compounds of the formulae (I)-(VII) and the salts thereof, the solvates thereof and the solvates of the salts thereof for use according to the invention, depending on their structure, may exist in stereoisomeric forms (enantiomers, diastereomers). The invention therefore also encompasses the enantiomers or diastereomers and respective mixtures thereof. The stereoisomerically uniform constituents can be isolated in a known manner from such mixtures of enantiomers and/or diastereomers.

If the compounds of formulae (I)-(VII) and the salts thereof, the solvates thereof and the solvates of the salts thereof for use according to the invention occur in tautomeric forms, the present invention encompasses all tautomeric forms.

Salts, solvates and solvates of the salts preferred for the purposes of the present invention are physiologically acceptable salts, solvates and solvates of the salts of the compounds of formulae (I)-(VII) for use according to the invention. Also encompassed, however, are salts, solvates and solvates of the salts which are themselves not suitable for pharmaceutical applications but can be for use, for example, for the isolation or purification of the compounds of formulae (I)-(VII).

Examples of pharmaceutically acceptable salts of the compounds of formula (I)-(VII) include salts of inorganic bases like ammonium salts, alkali metal salts, in particular sodium or potassium salts, alkaline earth metal salts, in particular magnesium or calcium salts; salts of organic bases, in particular salts derived from cyclohexylamine, benzylamine, octylamine, ethanolamine, diethanolamine, diethylamine, triethylamine, ethylenediamine, procaine, morpholine, pyrroline, piperidine, N-ethylpiperidine, N-methylmorpholine, piperazine as the organic base; or salts with basic amino acids, in particular lysine, arginine, ornithine and histidine.

Examples of pharmaceutically acceptable salts of the compounds of formulae (I) to (VII) for use according to the invention also include salts of inorganic acids like hydrochlorides, hydrobromides, sulfates, phosphates or phosphonates; salts of organic acids, in particular acetates, formates, propionates, lactates, citrates, fumarates, maleates, benzoates, tartrates, malates, methanesulfonates, ethanesulfonates, toluenesulfonates or benzenesulfonates; or salts with acidic amino acids, in particular aspartate or glutamate.

Solvates of the compounds of the formulae (I)-(VII) for use for the purposes of the invention refer to those forms of the compounds of formulae (I)-(VII) which in the solid or liquid state form a complex by coordination with solvent molecules. Hydrates are a specific form of solvates in which the coordination takes place with water.

The formulations of the invention may be provided as a stock solution, which is diluted with a liquid carrier composition such as WFI, saline or Ringer's lactate solution prior to administration to a subject. Alternatively, the formulation can be provided at a concentration of API that is suitable for administration without dilution.

Upon dilution with a pharmaceutically acceptable aqueous liquid carrier, the present formulations of the invention will not precipitate as parenterally injectable solution, preferably as i.v. injectable solution, when compared to a corresponding formulation not containing the modified cyclodextrin, particularly SBE-β-CD, and an organic and/or inorganic acid in accordance with the invention. The formulations of the invention do not require a surfactant in order to render the formulations suitable for dilution.

As used herein the expression “reconstitutable” in terms of a solid or similar expressions is taken to mean a solid capable of dissolution in an aqueous liquid medium to form a reconstituted liquid, wherein after dissolution the liquid medium is visibly clear.

A reconstitutable solid composition according to the present invention comprises an API in accordance with the invention, and a modified cyclodextrin, particularly SBE-β-CD, and an organic and/or an inorganic acid, particularly CA, and optionally at least one other pharmaceutical excipient.

For instance and not being limited thereto, a reconstitutable solid composition can be prepared by removal of the liquid medium from an aqueous liquid solution comprising an API in accordance with the invention, and a modified cyclodextrin, particularly SBE-β-CD, and an organic and/or an inorganic acid, particularly CA, and optionally at least one other pharmaceutical excipient.

A reconstitutable solid composition with the context of the invention will generally comprise 2-3% water. This composition is reconstituted with an aqueous based solution to form a liquid formulation containing an API in accordance with the invention, and a modified cyclodextrin, particularly SBE-β-CD, and an organic and/or an inorganic acid, particularly CA, and optionally at least one other pharmaceutical excipient that is administered by injection or infusion to a subject.

The liquid formulation used in the preparation of a reconstitutable solid composition may be prepared as described herein for the diluted or concentrated liquid formulations. A reconstitutable solid composition can be made to form a reconstituted liquid formulation that is or is not dilutable after the solid has been reconstituted with a predetermined amount of an aqueous liquid and at a predetermined temperature.

The reconstitutable composition is prepared according to any of the processes further described below. A liquid formulation of the invention is first prepared, then a reconstitutable solid composition is formed by e.g. lyophilisation (i.e. freeze-drying), spray drying, spray freeze-drying, vacuum-drying, antisolvent precipitation, ball milling, or various other processes utilizing supercritical or near supercritical fluids, or other methods known to those of ordinary skill in the art to make a powder or a solid suitable for reconstitution.

A reconstitutable solid composition in accordance with the invention can be a powder, glassy solid, porous solid, or particulate. The reconstitutable solid composition can be crystalline or amorphous.

As used in regards to the API-modified cyclodextrin containing compositions or formulations according to the invention, the term “dilutable” refers to a liquid formulation containing the modified cyclodextrin in accordance with the invention and an API, wherein the formulation can be further diluted (e.g. with WFI) at room temperature, e.g., ambient temperature such as a temperature of about 20° C.-28° C. without precipitation of the API while maintaining a clear solution at pH 4.0 when diluted to an API concentration of about 4.3-5.0 mg/mL.

In accordance with the present invention, temperature will have an effect upon the dilutability of a solution. In general, the determination of whether or not a solution is dilutable is made at approximately 25° C. or ambient temperature, e.g., 20° C.-28° C. A solution that is not dilutable at about 25° C. can be made dilutable with water at room temperature by dilution at an elevated temperature, such as >30° C., >40° C., >50° C. or higher. This heated dilution can be performed by diluting the first 25° C. solution with a heated solution or by mixing and heating two solutions which are initially at ambient temperature. Alternatively, the two solutions can be heated separately and then mixed.

Dilutability of an API-modified cyclodextrin containing solution according to the invention at ambient temperature is particularly important in the clinical setting wherein solutions are not typically heated prior to mixing. Accordingly, the present invention provides solutions of API that can be diluted at ambient temperature without the need of a surfactant, organic solvent, soap, detergent or other such compound.

As used herein, a “pharmaceutically acceptable liquid carrier” is any aqueous medium used in the pharmaceutical sciences for dilution or dissolution of parenteral formulations.

The invention also provides for a method of administering the API of the invention comprising the step of administering a liquid formulation comprising a modified cyclodextrin, an organic and/or an inorganic acid and optionally at least one other pharmaceutical excipient. The formulation can be parenterally administered, preferably intravenously, subcutaneously, intradermally, intraperitoneally, via implant, intramuscularly, or intrathecally.

Specific embodiments of the methods of the invention include those wherein: 1) the liquid formulation is administered by injection or infusion; 2) the method further comprises the earlier step of mixing the API of the invention with a modified cyclodextrin, an organic and/or an inorganic acid and optionally at least one other pharmaceutical excipient, in a solution to form the liquid formulation; 3) the method further comprises the step of diluting the liquid formulation in a pharmaceutically acceptable liquid carrier prior to administration; 4) the method comprises the step of forming the liquid formulation by mixing a liquid carrier with a reconstitutable solid composition comprising the API of the invention, a modified cyclodextrin, an organic and/or an inorganic acid and optionally at least one other pharmaceutical excipient; 5) the liquid formulation is further formulated as described herein.

The pharmaceutical formulations of the invention can be also administered orally.

The present invention also provides methods of preparing an API-modified cyclodextrin-based liquid formulation with an organic and/or an inorganic acid.

Another aspect of the invention provides a kit comprising an API-modified cyclodextrin-based liquid formulation with an organic and/or an inorganic acid.

In further embodiments, the invention is directed to a kit comprising: a breakable container; an infusion bag; and a reconstitutable solid composition of the invention, wherein said container contains the composition, and said infusion bag contains a diluent, preferably Ringer's lactate solution, and wherein said breakable container is placed directly inside said infusion bag suitably to allow said composition to be diluted by breaking said breakable container directly inside the diluent in said infusion bag.

### Lyophilisation

By the present invention, the aqueous stability of the API-modified cyclodextrin complexes is further enhanced through lyophilisation (i.e. freeze-drying). The modified cyclodextrins used in formulations according to the invention enable the finished lyophilised product to accommodate high levels of moisture without a detrimental effect on stability.

Generally, in aqueous intravenous and intramuscular formulations according to the invention, the API of the aforementioned formulae (I) to (VII) will be present at a concentration of from 3 mg/mL to 50 mg/mL, preferably 5 mg/mL to 30 mg/mL, more preferably from 5 mg/mL to 10 mg/mL. The modified cyclodextrin will be present in a molar ratio of API:modified cyclodextrin of from 1:1 to 1:10, preferably of from 1:1 to 1:3.

Thus, the formulations of the invention may be lyophilised (i.e. freeze dried) for storage prior to use, and made up with a suitable medium when required (it is reconstitution).

The formulations of the invention can be provided in liquid form or as a reconstitutable powder; e.g. a lyophlisate (freeze dried powder).

The invention also provides a pharmaceutical kit comprising a first container containing a liquid vehicle and a second container containing a reconstitutable solid pharmaceutical composition as described above. The liquid vehicle Ringer's lactate solution, or any other pharmaceutically acceptable aqueous liquid vehicles for the preparation of a liquid pharmaceutical compound.

With the context of the present invention, a complexation-enhancing agent can be added to the aqueous liquid formulation of the invention. A complexation-enhancing agent is a compound, or compounds, that enhance(s) the complexation of API with modified cyclodextrin of the invention. When the complexation-enhancing agent is present, the required ratio of API to modified cyclodextrin and organic and/or inorganic acid may need to be changed such that less modified cyclodextrin and/or organic and/or inorganic acid is required.

Suitable complexation enhancing agents include one or more pharmacologically inert water soluble polymers, hydroxy acids, and other organic compounds typically used in liquid formulations to enhance the complexation of a particular agent with cyclodextrins.

Suitable water soluble polymers include water soluble natural polymers, water soluble semisynthetic polymers (such as the water soluble derivatives of cellulose) and water soluble synthetic polymers. The natural polymers include polysaccharides such as inulin, pectins, algin derivatives and agar, and polypeptides such as casein and gelatin. The semi-synthetic polymers include cellulose derivatives such as methylcellulose, hydroxyethyl cellulose, hydroxypropyl cellulose, their mixed ethers such as hydroxypropyl methylcellulose and other mixed ethers such as hydroxyethyl ethylcellulose, hydroxypropyl ethylcellulose, hydroxypropyl methylcellulose phthalate and carboxymethylcellulose and its salts, especially sodium carboxymethylcellulose.

The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer). Suitable hydroxy acids include by way of example, and without limitation, citric acid, malic acid, maleic acid, methanesulphonic acid, lactic acid, and tartaric acid and others known to those of ordinary skill in the art.

A solubility-enhancing agent can be added to the aqueous liquid formulation of the invention. A solubility-enhancing agent is a compound, or compounds, that enhance(s) the solubility of API in the liquid formulation. When a complexation-enhancing agent is present, the ratio of API to modified cyclodextrin and organic and/or inorganic acid may need be changed such that less modified cyclodextrin and organic and/or inorganic acid is required.

Suitable solubility enhancing agents include one or more organic solvents, detergents, soaps, surfactants and other organic compounds typically used in parenteral formulations to enhance the solubility of a particular agent. Suitable organic solvents include, for example, ethanol, glycerin, polyethylene glycols, propylene glycol, poloxomers, polysorbates, glycofuroal, DMA, DMF. DMS, DMSO and others known to those of ordinary skill in the art.

It should be understood that compounds used in the pharmaceutical arts generally serve a variety of functions or purposes. Thus, if a compound of the formulae (I) to (VII) is mentioned only once or is used to define more than one term herein, its purpose or function should not be construed as being limited solely to that named purpose(s) or function(s). Although not necessary, the formulations of the present invention may include a preservative, antioxidant, buffering agent, acidifying agent, alkalizing agent, antibacterial agent, antifungal agent, antiviral agent, anti-inflammatory agent, solubility-enhancing agent, complexation enhancing agent, solvent, electrolyte, salt, water, glucose, stabilizer, tonicity modifier, antifoaming agent, oil, bulking agent, cryoprotectant, or a combination thereof.

As used herein, the term “alkalizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

As used herein, the term “acidifying agent” is intended to mean a compound used to provide an acidic medium for product stability. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid and nitric acid and others known to those of ordinary skill in the art.

As used herein, the term “antioxidant” is intended to mean an agent which inhibits oxidation and thus is used to prevent the deterioration of preparations by the oxidative process. Such compounds include by way of example and without limitation, acetone, sodium bisulfate, ascorbic acid, ascorbyl palmitate, citric acid, butylated hydroxyanisole, butylated hydroxytoluene, hydrophosphorous acid, monothioglycerol, propyl gallate, sodium ascorbate, sodium citrate, sodium sulfide, sodium sulfite, sodium bisulfite, sodium formaldehyde sulfoxylate, thioglycolic acid, sodium metabisulfite, EDTA (edetate), pentetate and others known to those of ordinary skill in the art.

As used herein, the term “buffering agent” is intended to mean a compound used to resist change in pH upon dilution or addition of acid or alkali.

Such compounds include, by way of example and without limitation, acetic acid, sodium acetate, adipic acid, benzoic acid, sodium benzoate, citric acid, maleic acid, monobasic sodium phosphate, dibasic sodium phosphate, lactic acid, tartaric acid, glycine, potassium metaphosphate, potassium phosphate, monobasic sodium acetate, sodium bicarbonate, sodium tartrate and sodium citrate anhydrous and dihydrate and others known to those of ordinary skill in the art.

As used herein, the term “stabilizer” is intended to mean a compound used to stabilize a therapeutic agent against physical, chemical, or biochemical process that would otherwise reduce the therapeutic activity of the agent. Suitable stabilizers include, by way of example and without limitation, albumin, sialic acid, creatinine, glycine and other amino acids, niacinamide, sodium acetyltryptophanate, zinc oxide, sucrose, glucose, lactose, sorbitol, mannitol, glycerol, polyethylene glycols, sodium caprylate and sodium saccharin and others known to those of ordinary skill in the art.

As used herein, the term “tonicity modifier” is intended to mean a compound or compounds that can be used to adjust the tonicity of the liquid formulation. Suitable tonicity modifiers include glycerin, lactose, mannitol, dextrose, sodium chloride, sodium sulfate, sorbitol, trehalose and others known to those or ordinary skill in the art. In one embodiment, the tonicity of the liquid formulation approximates that of the tonicity of blood or plasma.

As used herein, the term “antifoaming agent” is intended to mean a compound or compounds that prevents or reduces the amount of foaming that forms on the surface of the liquid formulation. Suitable antifoaming agents include by way of example and without limitation, dimethicone, simethicone, octoxynol and others known to those of ordinary skill in the art.

As used herein, the term “bulking agent” is intended to mean a compound used to add bulk to the reconstitutable solid and/or assist in the control of the properties of the formulation during preparation. Such compounds include, by way of example and without limitation, dextran, trehalose, sucrose, polyvinylpyrrolidone, lactose, inositol, sorbitol, dimethylsulfoxide, glycerol, albumin, calcium lactobionate, and others known to those of ordinary skill in the art.

As used herein, the term “cryoprotectant” is intended to mean a compound used to protect an active therapeutic agent from physical or chemical degradation during lyophilization. Such compounds include, by way of example and without limitation, dimethyl sulfoxide, glycerol, trehalose, propylene glycol, polyethylene glycol, and others known to those of ordinary skill in the art.

As used herein, the term “solubilizing agent” is intended to mean a compound used to assist and or increase the solubility of a compound going into solution. Such compounds include, by way of example and without limitation, glycerin, glycerol, polyethylene glycol, propylene glycol, ethanol, DMSO, DMS, DMF, DMA, glycofurol and others known to those of ordinary skill in the art.

The formulation of the invention can also include water, glucose or saline and combinations thereof. In particular embodiments, the formulations include water, saline, and glucose.

The chemical stability of the liquid formulations of the invention, in terms of a precipitate or gel forming, can be enhanced by adjusting the pH-1 of the liquid carrier. The chemical stability can also be enhanced by converting the liquid formulation to a solid or powder formulation.

The liquid formulation of the invention can be provided in an ampoule, prefilled syringe, bottle, bag, vial or other such container typically used for parenteral formulations.

The liquid formulation of the invention can be provided in a kit. The kit will comprise a first pharmaceutical composition comprising the modified-cyclodextrin formulation in accordance with the present invention and a second pharmaceutical composition comprising the API. The first and second formulations can be mixed and formulated as a liquid dosage form prior to administration to a subject. Either one or both of the first and second pharmaceutical compositions can comprise additional pharmaceutical excipients. The kit is available in various forms.

In a first kit, the first and second pharmaceutical compositions are provided in separate containers or separate chambers of a container having two or more chambers. The first and second pharmaceutical compositions may be independently provided in either solid or powder or liquid form. For example, the modified-cyclodextrin formulation in accordance with the present invention can be provided in a reconstitutable powder form and the API can be provided in powdered form.

According to one embodiment, the kit would further comprise a pharmaceutically acceptable liquid carrier used to suspend and dissolve the first and/or second pharmaceutical compositions. Alternatively, a liquid carrier is independently included with the first and/or second pharmaceutical composition. The liquid carrier, however, can also be provided in a container or chamber separate from the first and second pharmaceutical compositions. As above, the first pharmaceutical composition, the second pharmaceutical composition and the liquid carrier can independently comprise an antioxidant, a buffering agent, an acidifying agent, saline, glucose, an electrolyte, another therapeutic agent, an alkalizing agent, solubility enhancing agent or a combination thereof. The liquid formulation of the invention can be provided as a dosage form including a pre-filled vial, pre-filled bottle, pre-filled syringe, pre-filled ampoule, or plural ones thereof. Generally, a pre-filled container will contain at least a unit dosage form of the API.

Specific embodiments of the kit include those wherein: 1) the first and second pharmaceutical compositions are contained in separate containers or separate chambers of a container having two or more chambers; 2) the kit further comprises a separate pharmaceutically acceptable liquid carrier; 3) a liquid carrier is included with the first and/or second pharmaceutical composition; 4) containers for the pharmaceutical compositions are independently at each occurrence from an evacuated container, a syringe, bag, pouch, ampule, vial, bottle, or any pharmaceutically acceptable device known to those skilled in the art for the delivery of liquid formulations; 5) the first pharmaceutical composition and/or second pharmaceutical composition and/or liquid carrier further comprises an antioxidant, a buffering agent, an acidifying agent, a solubilizing agent, a complexation enhancing agent, saline, dextrose, lyophilizing aids (for example, bulking agents or stabilizing agents), an electrolyte, another therapeutic agent, an alkalizing agent, or a combination thereof; 6) the kit is provided chilled; 8) the liquid carrier and/or chamber has been purged with a pharmaceutically acceptable inert gas to remove substantially all of the oxygen dissolved in the liquid carrier; 9) the chambers are substantially free from oxygen; 10) the liquid carrier further comprises a buffering agent capable of maintaining a physiologically acceptable pH; 11) the chambers and solutions are sterile.

### Processes

In other aspects of the invention the processes for:
i) preparation/manufacture of a pre-lyo formulation solution,
ii) preparation/manufacture of a reconstitutable solid composition (e.g. a lyophilisate),
iii) preparation/manufacture of a pharmaceutical formulation,
iv) preparation/manufacture of an injectable aqueous solution, are provided.

### Process Formulation

Another aspect of the invention provides a process for the manufacture of a formulation of the invention comprising the steps of:
i) providing means for mixing, preferably a mixing tank,
ii) maintaining bulk solution temperature at approx. 50° C. by heating means, preferably by using a heat mixing jacket,
iii) adding approx. 60% w/v water for injection, preferably add hot 60% w/v water for injection at approx. 60° C.,
iv) maintaining bulk solution temperature at a range of 48-55° C., preferably 49-52° C., most preferred at 50° C., whereby 50° C. is the target temperature,
v) adding an organic and/or an inorganic acid in accordance with the invention and mixing the solution, preferably mixing at least for 3 minutes, more preferred at least for 4 minutes, most preferred at least for 5 minutes until dissolved,
vi) adding a modified cyclodextrin in accordance with the invention and mixing the solution, preferably mixing at least for 20 minutes, more preferred at least for 25 minutes, most preferred at least for 30 minutes until dissolved,
vii) adding an API in accordance with the invention and ensure that bulk solution temperature is at a range of 48-55° C., preferably 49-52° C., most preferred at 50° C., whereby 50° C. is the target temperature,
viii) and mixing the solution obtained under step vii) until visually dissolution is observed, and QS to 100% bulk volume using water for injection at room temperature, thereby maintaining bulk solution at 25-35° C., preferably at 29-35° C., most preferred at 34-35° C., whereby 34-35° C. is the target temperature,
ix) optionally take in process sample(s) to monitor pH or for using other assays,
x) set up particulate reduction filter, preferably a 0.45 am particulate reduction filter, on mixing means, preferably on mixing tank,
xi) ensuring transfer line temperature is at 25-35° C., preferably at 29-35° C., most preferred at 34-35° C., whereby 34-35° C. is the target temperature,
xii) transferring product of step xi) immediately to filling room, as soon as bulk solution reached a temperature at 34-35° C. as target temperature,
xiii) filtering bulk solution of step xii) through 0.2 am filter, preferably through two 0.2 μm filter, whereby more preferably said filter is a Polyvinylidene difluoride membrane (PVDF)
xiv) optionally perform offline filter testing,
xv) fill bulk solution,
xvi) lyophilize product obtained under step xv),
xvii) optionally decontaminate lyophilized product obtained under step xvi.

In a more preferred aspect of the invention, the above described steps viii) to xv), the process temperature is continuously held at 34-35° C., in order to avoid any API precipitation during process.

In view of the above, it is another surprising finding of the invention that through maintaining the temperature at 34-35° C. for the above described steps viii) to xv), subsequent filtration and lyophilization is possible without precipitation of the API contained in the bulk solution.

In a preferred aspect, the above process comprises the following exemplary steps:
i) providing a mixing tank,
ii) maintaining bulk solution temperature at approx. 50° C. by heat mixing jacket,
iii) adding approx. 60% w/v water for injection, preferably add hot 60% w/v water for injection at approx. 60° C.,
iv) maintaining bulk solution temperature at a range of 48-55° C., preferably 49-52° C., most preferred at 50° C., whereby 50° C. is the target temperature,
v) adding 600 g (1%) citric acid and mixing the solution, preferably mixing at least for 3 minutes, more preferred at least for 4 minutes, most preferred at least for 5 minutes until dissolved,
vi) adding 12 kg SBE-β-CD and mixing the solution, preferably mixing at least for 20 minutes, more preferred at least for 25 minutes, most preferred at least for 30 minutes until dissolved,
vii) adding 1.92 kg of a compound of formula (I) as API (corrected amount for water content) and ensure that bulk solution temperature is at a range of 48-55° C., preferably 49-52° C., most preferred at 50° C., whereby 50° C. is the target temperature,
viii) and mixing the solution obtained under step vii) until visually dissolution is observed, and QS to 100% bulk volume using water for injection at room temperature, thereby maintaining bulk solution at 25-35° C., preferably at 29-35° C., most preferred at 34-35° C., whereby 34-35° C. is the target temperature,
ix) optionally take in process sample(s) to monitor pH or for using other assays,
x) set up particulate reduction filter, preferably a 0.45 am particulate reduction filter, on mixing means, preferably on mixing tank,
xi) ensuring transfer line temperature is at 25-35° C., preferably at 29-35° C., most preferred at 34-35° C., whereby 34-35° C. is the target temperature,
xii) transferring product of step xi) immediately to filling room, as soon as bulk solution reached a temperature at 34-35° C. as target temperature,
xiii) filtering bulk solution of step xii) through 0.2 am filter, preferably through two 0.2 am filter, whereby more preferably said filter is a Polyvinylidene difluoride membrane (PVDF)
xiv) optionally perform offline filter testing,
xv) fill bulk solution,
xvi) lyophilize product obtained under step xv),
xvii) optionally decontaminate lyophilized product obtained under step xvi.

In another aspect of the invention, the solution obtained under step xv) above can be lyophilized for at least 98 hours, so to obtain a reconstitutable solid composition in accordance with the invention.

In another aspect of the invention, the lyophilisation takes place by freeze drying with optimized cycles in terms of temperature and pressure frequency according to manufacturer's lyophilization recipe including loading, freezing, primary drying, secondary drying, and unloading: Partial stopper the vials. Load vials into freeze-drier. Commence cycle. Stopper vials under nitrogen. Unload vials from the freeze-drier.

Process Reconstitution In another aspect of the invention, the lyophilisate described above can be reconstituted by the addition of different suitable reconstitution media selected from a group consisting of Type I water, 0.9% NaCl solution, a 5% dextrose solution, WFI, and Ringer's lactate solution.

With the above general context of the invention, further specific aspects of the invention are represented by the below consecutively numbered embodiments and the adjacent embodiments:
1. A formulation comprising a compound selected from a group of compounds consisting of the formulae (I) to (VII):

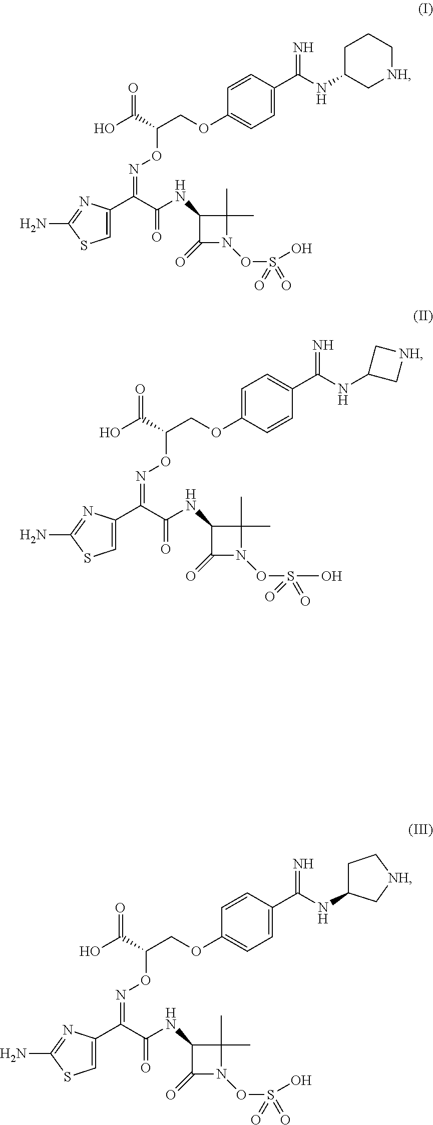 [SMILES: CC1(C)[C@H](NC(=O)/C(=N\O[C@@H](COc2ccc(C(=N)N[C@@H]3CCCNC3)cc2)C(=O)O)c2csc(N)n2)C(=O)N1OS(=O)(=O)O ; CC1(C)[C@H](NC(=O)/C(=N\O[C@@H](COc2ccc(C(=N)NC3CNC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCNC3)cc2)C(=O)O)c2csc(N)n2)C(=O)N1OS(=O)(=O)O]

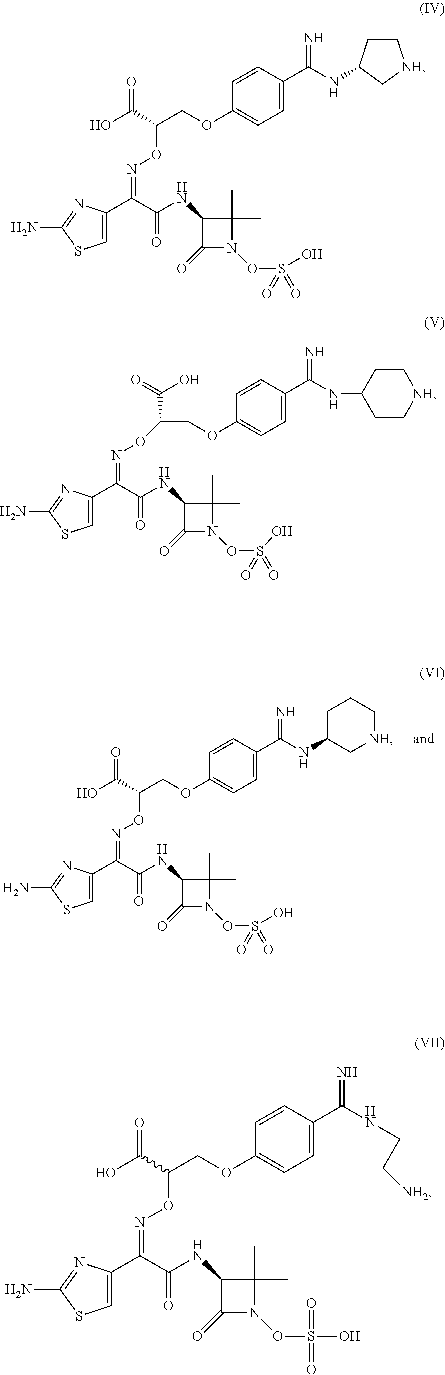 [SMILES: CC1(C)[C@H](NC(=O)/C(=N\O[C@@H](COc2ccc(C(=N)N[C@@H]3CCNC3)cc2)C(=O)O)c2csc(N)n2)C(=O)N1OS(=O)(=O)O ; CC1(C)[C@H](NC(=O)/C(=N\O[C@@H](COc2ccc(C(=N)NC3CCNCC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCCNC3)cc2)C(=O)O)c2csc(N)n2)C(=O)N1OS(=O)(=O)O ; "and" ; CC1(C)[C@H](NC(=O)/C(=N\OC(COc2ccc(C(=N)NCCN)cc2)C(=O)O)c2csc(N)n2)C(=O)N1OS(=O)(=O)O]

or the salts thereof, the solvates thereof or the solvates of the salts thereof,
and further comprising
a) an organic acid selected from the group comprising citric acid, tartaric acid, malic acid, maleic acid, methanesulphonic acid, ascorbic acid, adipic acid, aspartatic acid, benzenesulfonic acid, glucoheptonic acid, D-gluconic acid, L-glutamic acid, lactic acid, L-Lysine, saccharin; and/or
b) an inorganic acid selected from the group comprising hydrochloric acid, sulfuric acid, phosphoric acid, and nitric acid; and
c) a modified cyclodextrin in aqueous solution, preferably in quantum satis aqueous solution,
   wherein
   i) said compound of the formulae (I)-(VII) has a concentration in the range of 1-5% w/v, with the proviso that at least an organic acid according to a) is used, and wherein said organic acid has a concentration in the range of 0.25-4% w/v, or
   ii) wherein said compound of the formulae (I)-(VII) has a concentration in the range of 1-15% w/v, with the proviso that only an inorganic acid according to b) is used, and
   wherein either for i) or ii) said inorganic acid has a concentration in the range of 0.25-6% w/v, and
   wherein either for i) or ii) said modified cyclodextrin has a concentration in the range of 10-40% w/v in said aqueous solution, and
   wherein either for i) or ii) said formulation has a pH in the range of 1.25 to 2.8.

With the above context of embodiment 1 the person skilled in the art will immediately recognise that three basic alternatives are given for the formulation in terms of comprising organic/inorganic acid(s) in addition to at least one of the cited compounds (I) to (VII) and a modified cyclodextrin in aqueous solution; namely either 1) only an organic acid according to a) or 2) an organic acid according to a) and an inorganic acid according to b) or 3) only an inorganic acid according to b).

Depending on the presence of an organic/inorganic acid, the concentration ranges of the compound of the formulae (I)-(VII) differ in that, in case of the presence of an organic acid; i.e. without or with an additional inorganic acid, the said concentration of the compound of the formulae (I)-(VII) ranges from 1-5% w/v. Whereas in the absence of an organic acid according to a); i.e. with the only presence of an inorganic acid, the said concentration of a compound of the formulae (I)-(VII) ranges from 1-15% w/v.

Whatever the case may be, when an organic acid is present in the formulation of embodiment 1, the respective concentration of organic acid ranges from 0.25-4% w/v. When an inorganic acid is present in the formulation of embodiment 1, the respective concentration of inorganic acid ranges from 0.25-6%.

In another embodiment of the invention, in the above mentioned formulation said compound (I)-(VII) has a concentration in the range of 2-4% w/v, and wherein said organic acid has a concentration in the range of 0.5-3% w/v, and wherein said inorganic acid has a concentration in the range of 0.5-3% w/v, and wherein said modified cyclodextrin has a concentration in the range of 15-30% w/v in said q.s. aqueous solution, and wherein said formulation has a pH in the range of 2 to 2.5.

In yet another embodiment of the invention, in the above mentioned formulation said compound (I)-(VII) has a concentration in the range of 3-4% w/v, and wherein said organic acid has a concentration in the range of 1-2% w/v, and wherein said inorganic acid has a concentration in the range of 2-2.75% w/v, and wherein said modified cyclodextrin has a concentration in the range of 20-25% w/v in said q.s. aqueous solution, and wherein said formulation has a pH in the range of 2 to 2.3.
2. The formulation according to embodiment 1 and the above mentioned adjacent embodiments, wherein said compound is a compound according to formula (I).
3. The formulation according to embodiment 1 or embodiment 2 and the above mentioned adjacent embodiments, wherein said modified cyclodextrin in aqueous solution is selected from a group comprising α-cyclodextrin, β-cyclodextrin, γ-cyclodextrin or a modified derivative thereof.
   With the above embodiment 3 it should be noted that the said modified cyclodextrin or a modified derivative thereof is to be meant for being in quantum satis (q.s.) aqueous solution.
4. The formulation according to embodiment 3 and the above mentioned adjacent embodiments, wherein said β-cyclodextrin is selected from a group comprising carboxymethyl-β-cyclodextrin, carboxymethyl-ethyl-β-cyclodextrin, diethyl-β-cyclodextrin, dimethyl-β-cyclodextrin, glucosyl-β-cyclodextrin, hydroxybutenyl-β-cyclodextrin, hydroxyethyl-β-cyclodextrin, hydroxypropyl-β-cyclodextrin, maltosyl-β-cyclodextrin, methyl-β-cyclodextrin, random methyl-β-cyclodextrin, sulfobutylether-β-cyclodextrin or a modified derivative thereof.
5. The formulation according to any of the embodiments 3 to 4 and the above mentioned adjacent embodiments, wherein said β-cyclodextrin is selected from a group comprising hydroxypropyl-β-cyclodextrin, methyl-β-cyclodextrin, random methyl-β-cyclodextrin, sulfobutylether-β-cyclodextrin or a modified derivative thereof.
6. The formulation according to any of the embodiments 3 to 5 and the above mentioned adjacent embodiments, wherein said β-cyclodextrin is selected from a group comprising hydroxypropyl-β-cyclodextrin and sulfobutylether-β-cyclodextrin or a modified derivative thereof.
7. The formulation according to any of the embodiments 3 to 6 and the above mentioned adjacent embodiments, wherein said β-cyclodextrin is sulfobutylether-β-cyclodextrin or a modified derivative thereof.
8. The formulation according to embodiment 3 and the above mentioned adjacent embodiments, wherein said γ-cyclodextrin is hydroxypropyl-γ-cyclodextrin or a modified derivative thereof.
9. The formulation according to any of the preceding embodiments, wherein said organic acid is selected from a group comprising citric acid, tartaric acid, malic acid, maleic acid, methanesulphonic acid, ascorbic acid, L-Lysine, and saccharin.
10. The formulation according to any of the preceding embodiments, wherein said organic acid is selected from a group comprising citric acid, tartaric acid, ascorbic acid, and saccharin.
11. The formulation according to any of the preceding embodiments, wherein said inorganic acid is selected from a group comprising hydrochloric acid, sulfuric acid, and phosphoric acid.
12. The formulation according to any of the preceding embodiments, wherein said inorganic acid is selected from a group comprising sulfuric acid and phosphoric acid.
13. The formulation according to any of the preceding embodiments, wherein said formulation is further comprising a solubilizing agent, antioxidant, buffering agent, acidifying agent, complexation enhancing agent, saline, dextrose, lyophilizing aid, bulking agent, stabilizing agent, electrolyte, another therapeutic agent, alkalizing agent, antimicrobial agent, antifungal agent, antiviral agent, anti-inflammatory agent or a combination thereof.
14. The formulation according to embodiment 13, wherein said stabilizing agent is selected from a group comprising sugars and polymers.

In yet another embodiment of the invention, in the above mentioned formulations said formulation is further comprising a solubilizing agent, antioxidant, buffering agent, acidifying agent, complexation enhancing agent, saline, dextrose, lyophilizing aid, bulking agent, stabilizing agent, electrolyte, another therapeutic agent, alkalizing agent, antibacterial agent, antifungal agent, antiviral agent, anti-inflammatory agent, antiparasitic agent, antimycotic agent, antimycobacterial agent, intestinal antiinfective agent, antimalaria agent, anti-inflammatory agent, anti-allergic agent, analgesic drug, anaesthetic drug, immunomodulator, immune suppressive agent, monoclonal antibodies, anti-neoplastic drug, anti-cancer drug, anti-emetic, anti-depressive, anti-psychotic, anxiolytic, anti-convulsive, HMG CoA reductase inhibitor and other anti-cholesterol agents, anti-hypertensives, insulins, oral anti-diabetics, proton pump inhibitors, oral or parenteral anti-coagulants, diuretics, digoxins, broncho dialators, anti-arrythmics, vasopressors, steroids and derivatives and combinations thereof.

Specifically, in yet another embodiment of the invention, the formulations comprising a compound according to the formulae (I)-(VII) according to the present invention may be used in combination with at least one beta-lactamase-inhibitor (BLI), which may be administered separately. The BLI may also be formulated in a similar fashion as the APIs of the formulae (I)-(VII) are formulated in accordance with the invention.

As used herein, a suitable BLI may be selected from the group comprising: clavulanic acid, tazobactam, sulbactam and other BLIs belonging to the groups of lactam inhibitors, DABCO inhibitors, BATSI inhibitors and/or metallo-beta-lactamase inhibitors. These BLIs together with the formulations according to the present invention may be administered in methods of treatment or prevention and are compounds for the use in the treatment of prophylaxis of a subject having an infection caused by Gram-negative bacteria that produce at least one or more class A or class D extended-spectrum beta-lactamase (ESBL) and at least one additional beta-lactamase selected from the groups of class C AmpC beta-lactamases and/or at least one class A, class B, class C and class D carbapenemase.

In yet another embodiment of the invention, in the above mentioned formulations said stabilizing agent is selected from a group comprising sugars and polymers.

In accordance with the invention, on basis of the aforementioned formulations through further processing such as lyophilisation, a reconstitutable solid composition can be obtained providing for the technical advantages as recited in the introduction portion above.

Thus, the invention further provides for the following consecutively numbered embodiments and the adjacent embodiments thereon:
15. A solid composition, wherein said solid composition is comprising at least one compound according to the formulae (I)-(VII) as defined in embodiment 1, and at least one modified cyclodextrin with a concentration of up to 95% w/w as defined in any of the embodiments 1, and 3 to 8, and at least one organic acid with a concentration of up to 20% w/w as defined in any of the embodiments 1, 9 and 10, and/or at least one inorganic acid with a concentration of up to 25% w/w as defined in any of the embodiments 1, and 11-12.
16. A solid composition according to embodiment 15, wherein said modified cyclodextrin has a concentration within the range of 60-90% w/w, and said organic acid has a concentration within the range of 1-10% w/w, and said inorganic acid has a concentration within the range of 1-15% w/w.
17. A solid composition according to any of the embodiments 15 to 16, wherein said modified cyclodextrin has a concentration within the range of 75-85% w/w, and said organic acid has a concentration within the range of 2-6% w/w, and said inorganic acid has a concentration within the range of 2-6% w/w.
18. A solid composition according to any of the embodiments 15 to 17, wherein said solid composition may be stored in sealed glass vials, and wherein said solid composition is further characterized by a stability of said compound according to the formulae (I)-(VII) over 12 months at 25° C./60% relative humidity, or 2-8° C. ambient temperature, or at −20° C. ambient temperature storing condition.
19. A solid composition according to any of the embodiments 15 to 18, wherein said solid composition is further characterized by an in-use stability of said compound according to the formulae (I)-(VII) up to 24 hours at room temperature.

With the above context of the embodiments 15 to 19, in another aspect of the invention said solid composition is a reconstitutable solid composition.

In another adjacent embodiment to the embodiments 15 to 19, said solid composition is obtainable from the formulation according to the above embodiments 1 to 14.

With this context of “in-use stability” of the compounds (I)-(VII) as API in accordance of the invention, the person skilled in the art is well aware that a continued integrity of medicinal products (here the reconstitutable solid composition and/or the aqueous injectable formulation of the invention) in multidose containers/bags after the first opening is an important quality issue. The person skilled in the art knows that the aqueous injectable formulation of the invention may be provided to the patient in methods of parenteral administration, preferably by i.v. injection, in multiple dosage forms.

While this principle of in-use stability is acknowledged in the Ph. Eur. and EU Guidelines, specific guidance for test design and conduct of studies to be undertaken to define in-use shelf life in a uniform fashion is provided by the “Note for guidance on in-use stability testing of human medicinal products” as published by the European Agency for the Evaluation of Medicinal Products. This document attempts to define a framework for selection of batches, test design, test storage conditions, test parameters, test procedures etc., taking into consideration the broad range of products concerned.
20. A solid composition according to any of the embodiments 15 to 19, wherein said solid composition is further characterized by a residual water content of 2-3% w/w.
21. A solid composition according to any of the embodiments 15 to 20 for use in parenteral administration to a subject in need thereof.
22. A solid composition according to embodiment 21, wherein said parenteral administration is intravenous injection.
23. A solid composition according to any of the embodiments 15 to 20 for use in oral administration to a subject in need thereof.
24. A solid composition for use in oral administration to a subject in need thereof according to embodiment 23, whereby said solid composition is formulated as tablet or capsule.
25. A solid composition according to any of the embodiments 15 to 24 for use in a method of treating and/or preventing bacterial infections.
26. A solid composition for the use in a method of treating and/or preventing bacterial infections according to embodiment 25, wherein said bacterial infections are Gram-negative bacterial infections.

With the above context of the embodiments 20 to 26, in another aspect of the invention said solid composition is a reconstitutable solid composition.

In accordance with the invention, upon reconstution of the above defined solid compositions with a suitable medium, the below defined pharmaceutical formulations can be obtained, providing to the technical advantages as recited in the introduction portion above.

A pharmaceutical formulation that is obtained upon reconstution of the above defined solid compositions with a suitable medium that may comprise water for injection, an organic acid, e.g., citric acid, or an inorganic acid, a modified cyclodextrin as defined herein. In a specific embodiment, the pharmaceutical formulation comprises water for injection, citric acid (7.5-11 mg/ml (particularly, 10 mg/ml), captisol (155-220 mg/ml, particularly, 200 mg/ml) and an active ingredient according to the present invention (25-35 mg/ml; particularly 32 mg/ml). In a very particular embodiment of the invention, the pharmaceutical formulation comprises water for injection, citric acid (10 mg/ml), captisol (200 mg/ml) and an active ingredient according to the present invention (particularly 32 mg/ml).

Thus, the invention also provides for the below consecutively numbered embodiments and adjacent embodiments thereon:
27. A pharmaceutical formulation obtainable from the solid composition according to any of the embodiments 15 to 20, in particular obtained by lyophilization.
28. A pharmaceutical formulation according to embodiment 27, wherein said formulation is obtainable from said solid composition upon reconstitution by making up a lyophilized formulation in a suitable aqueous medium.

Another adjacent embodiment of the invention to embodiment 28, is the provision of a pharmaceutical formulation according to embodiment 28, wherein said formulation comprises a compound according to any of formulae (I) to (VII) at 6-15%, preferably at 13.2%; Captisol at 60-95%, preferably at 82%, and citric acid at 2-10%, preferably at 4.1%. Therefore, another embodiment of embodiment 28 is the provision of a pharmaceutical formulation according to embodiment 28, wherein said formulation comprises a compound according to any of formulae (I) to (VII) at 13.2%; Captisol at 82%, and citric acid at 4.1%.

Another adjacent embodiment of the invention to embodiment 28, is the provision of a pharmaceutical formulation according to embodiment 28, wherein said pharmaceutical formulation is further characterized by an in-use stability of said compound according to the formulae (I)-(VII) in the reconstituted aqueous solution for over 24 hours at room temperature.

With this context of “in-use stability” of the compounds (I)-(VII) as API in accordance of the invention, the person skilled in the art is well aware that a continued integrity of medicinal products (here the reconstitutable solid composition and/or the aqueous injectable formulation of the invention) in multidose containers/bags after the first opening is an important quality issue. The person skilled in the art knows that the aqueous injectable formulation of the invention may be provided to the patient in methods of parenteral administration, preferably by i.v. injection, in multiple dosage forms.

While this principle of in-use stability is acknowledged in the Ph. Eur. and EU Guidelines, specific guidance for test design and conduct of studies to be undertaken to define in-use shelf life in a uniform fashion is provided by the “Note for guidance on in-use stability testing of human medicinal products” as published by the European Agency for the Evaluation of Medicinal Products. This document attempts to define a framework for selection of batches, test design, test storage conditions, test parameters, test procedures etc., taking into consideration the broad range of products concerned.
29. A pharmaceutical formulation according to embodiment 28, wherein said suitable aqueous medium is selected from a group comprising Ringer's lactate solution, water, saline solution, 5% dextrose solution, or water for injection.
30. A pharmaceutical formulation according to any of the embodiments 27 to 29, wherein said pharmaceutical formulation is further comprising phosphate buffer/saline mixed solution for pH adjustment towards a range between 4.0 and 4.5.
31. A pharmaceutical formulation as defined in any of the embodiments 27 to 30, wherein said pharmaceutical formulation is visibly clear at pH 4.0-4.5 without any precipitated compound of the formulae (I)-(VII) as defined in claim 1, upon dilution in aqueous media as defined the embodiments 28 to 29 at room temperature.

In another embodiment of the invention, upon further dilution of the pharmaceutical formulations obtained as described in the embodiment 31, aqueous injectable solutions can be obtained which provide for the technical advantages as recited in the introduction portion above.

Thus, in another aspect the invention provides for the below consecutively numbered embodiments and adjacent embodiments thereto:
32. An aqueous injectable formulation comprising a compound of the formulae (I)-(VII) as defined in embodiment 1 and 2, a modified cyclodextrin as defined in the embodiments 1-8, an organic and/or inorganic acid as defined in the embodiments 1-12, and water, wherein said aqueous injectable formulation is having a pH within the range between 4.0 and 4.5.
33. An aqueous injectable formulation comprising a compound of the formulae (I)-(VII) as defined in the embodiments 1 and 2, sulfobutylether-β-cyclodextrin as defined in the embodiments 3-7, citric acid as defined in the embodiments 1-10, and water, wherein said aqueous injectable formulation is having a pH within the range between 4.0 and 4.5.
34. An aqueous injectable formulation according to the embodiment 32 and 33, wherein said aqueous injectable formulation comprises a compound of the formula (I) as defined in the embodiments 1 and 2 in an amount from about 1.5 to about 8 mg/mL of formulation, sulfobutylether-3-cyclodextrin in an amount within the range from about 15 to about 40 mg/mL, citric acid in an amount within the range from about 0.5 to about 4 mg/mL, and Ringer's lactate solution q.s.
35. The aqueous injectable formulation according to the embodiments 32-34, wherein after said formulation has been injected into an infusion bag, the formulation and the infusate have been admixed, and the resulting admixture has been allowed to stand for up to 24 hours at room temperature, no compound of the formula (I)-(VII) precipitate is visible.
36. Use of an aqueous injectable formulation according to any one of embodiments 32-35 for the manufacture of a medicament for the treatment and/or prophylaxis of bacterial infections.
37. Use of an aqueous injectable formulation according to any one of embodiments 32-35 for the manufacture of a medicament for the treatment and/or prophylaxis of Gram-negative bacterial infections.
38. Use of an aqueous injectable formulation according to any one of embodiments 32-35 in combination with at least one further active compound in the manufacture of a medicament.
39. Use of an aqueous injectable formulation according to any one of embodiments 32-35 in combination with at least one further active compound in the manufacture of a medicament, wherein said active compound is a beta-lactamase inhibitor.
40. Use of an aqueous injectable formulation according to embodiment 39, wherein said beta-lactamase inhibitor is selected from a group comprising carbapenems, diazabicyclooctane inhibitors, transition state analog inhibitors and/or metallo-beta-lactamase inhibitors.
41. Use of an aqueous injectable formulation according to embodiment 40, wherein said beta-lactamase inhibitor is selected from a group comprising clavulanic acid, tazobactam, sulbactam, DABCO inhibitors, BATSI inhibitors.
42. Use of an aqueous injectable formulation according to any one of embodiments 32-35 for treating and/or preventing bacterial infections.
43. Use of an aqueous injectable formulation according to any one of embodiments 32-35 for treating and/or preventing Gram-negative bacterial infections.
44. A method for administering an aqueous injectable formulation as defined in the embodiments 32-35 to a patient in need of antimicrobial treatment, which comprises administering to a patient in need of said treatment the formulation as defined in any of the embodiments 32-35.
45. The method as defined in embodiment 44, wherein the aqueous injectable formulation is administered intravenously.

The formulations, reconstitutable solid compositions, pharmaceutical compositions, and aqueous injectable formulations with compounds according to formulae (I)-(VII) as active pharmaceutical ingredients (API(s)) according to the invention are particularly useful in human and veterinary medicine for the prophylaxis and treatment of local and systemic infections which are caused for example by the following pathogens or by mixtures of the following pathogens:

Aerobic Gram-positive bacteria including but not limited to Staphylococcus spp. (S. aureus), Streptococcus spp. (S. pneumoniae, S. pyogenes, S. agalactiae, Streptococcus group C and G) as well as Bacillus spp. and Listeria monocytogenes;

Aerobic Gram-negative bacteria: Enterobacteriaceae including but not limited to Escherichia spp. (E. coli), Citrobacter spp. (C. freundii, C. diversus), Klebsiella spp. (K. pneumoniae, K. oxytoca), Enterobacter spp. (E. cloacae, E. aerogenes), Morganella morganii, Hafnia alvei, Serratia spp. (S. marcescens), Proteus spp. (P. mirabilis, P. vulgaris, P. penneri), Providencia spp. (P. stuartii, P. rettgeri), Yersinia spp. (Y. enterocolitica, Y. pseudotuberculosis), Salmonella spp., Shigella spp. and also non-fermenters including but not limited to Pseudomonas spp. (P. aeruginosa), Burkholderia spp. (B. cepacia), Stenotrophomonas maltophilia, and Acinetobacter spp. (A. baumannii, Acinetobacter gen. sp. 13TU, Acinetobacter gen. sp. 3) as well as Bordetella spp. (B. bronchiseptica), Moraxella catarrhalis and Legionella pneumophila; furthermore, Aeromonas spp., Haemophilus spp. (H. influenzae), Neisseria spp. (N. gonorrhoeae, N. meningitidis) as well as Alcaligenes spp. (including A. xylosoxidans), Pasteurella spp. (P. multocida), Vibro spp. (V. cholerae), Campylobacterjejuni and Helicobacter pylori.

Moreover, the antibacterial spectrum also covers strictly anaerobic bacteria including but not limited to Bacteroides spp. (B. fragilis), Peptostreptococcus spp. (P. anaerobius), Prevotella spp., Brucella spp. (B. abortus), Porphyromonas spp., and Clostridium spp. (Clostridium perfringens).

The above listing of pathogens is merely exemplary and in no way to be regarded as limiting. Examples of diseases which may be caused by the said pathogens and which may be prevented, improved or cured by the formulations, reconstitutable solid compositions, pharmaceutical compositions, and aqueous injectable formulations with compounds according to formulae (I)-(VII) as pharmaceutically active substances according to the invention are, for example:

Respiratory tract infections such as lower respiratory tract infections, lung infection in cystic fibrosis patients, acute exacerbation of chronic bronchitis, community aquired pneumonia (CAP), nosocomial pneumonia (including ventilator-associated pneumonia (VAP)), diseases of the upper airways, diffuse panbronchiolitis, tonsillitis, pharyngitis, acute sinusitis and otitis including mastoiditis; urinary tract and genital infections for example cystitis, uretritis, pyelonephritis, endometritis, prostatitis, salpingitis and epididymitis; ocular infections such as conjunctivitis, corneal ulcer, iridocyclitis and post-operative infection in radial keratotomy surgery patients; blood infections, for example septicaemia; infections of the skin and soft tissues, for example infective dermatitis, infected wounds, infected burns, phlegmon, folliculitis and impetigo; bone and joint infections such as osteomyelitis and septic arthritis; gastrointestinal infections, for example dysentery, enteritis, colitis, necrotising enterocolitis and anorectal infections; intraabdominal infections such as typhoid fever, infectious diarrhea, peritonitis with appendicitis, pelviperitonitis, and intra-abdominal abscesses; infections in the oral region for example infections after dental operations; other infections for example, meliodosis, infectious endocarditis, hepatic abscesses, cholecystitis, cholangitis, mastitis as well as meningitis and infections of the nervous systems.

In addition to humans, bacterial infections can also be treated in animals, such as primates, pigs, ruminants (cow, sheep, goat), horses, cats, dogs, poultry (such as hen, turkey, quail, pigeon, ornamental birds) as well as productive and ornamental fish, reptiles and amphibians.

Yet another aspect of the invention provides for the use of an aqueous injectable formulation as defined above in combination with at least one further active compound in the manufacture of a medicament, wherein said active compound is a beta-lactamase inhibitor.

Thus, another aspect of the invention provides for the use of an aqueous injectable formulation as defined above in combination with at least one further active compound in the manufacture of a medicament, wherein said beta-lactamase inhibitor is selected from a group comprising lactam inhibitors, diazabicyclooctane inhibitors, transition state analog inhibitors and/or metallo-beta-lactamase inhibitors.

Another aspect of the invention provides for the use of an aqueous injectable formulation as defined above in combination with at least one further active compound in the manufacture of a medicament, wherein said compound is selected from the group compromising oxapenams (e.g. clavulanic acid and the like), penam sulfones (e.g. tazobactam, sulbactam, AAI-101 and the like), bridged monobactams (e.g. BAL29880, MK-8712 and the like), monobactams (e.g. aztreonam, carumonam, tigemonam, BAL30072 and the like), cephem sulfones (e.g 7-alkylidenecephalosporin sulfone and the like), carbapenems (e.g. imipenem, meropenem, ertapenem, doripenem and the like), penems (e.g. LK-157 and the like), diazabicyclooctane inhibitors (e.g. avibactam, relebactam, zidebactam, OP0595, WCK 4234, WCK 5153, CB-618 and the like), transition state analog BLIs (boronates, phosphonates, e.g. vaborbactam, MG96077 and the like), and/or metallo-beta-lactamase inhibitors (e.g. captopril and the like).

Yet further embodiments of the invention can be derived from the below consecutively numbered embodiments and adjacent embodiments thereto:
46. A method for administering an aqueous injectable formulation as defined in the embodiments 32-35 to a patient in need of antimicrobial treatment, which comprises administering to a patient in need of said treatment the formulation as defined in any of the embodiments 32-35.
47. The method as defined in embodiment 44, wherein the aqueous injectable formulation is administered intravenously.
48. A kit comprising:
   a breakable container,
   an infusion bag,
   wherein said container contains the reconstitutable solid composition as defined in any of the embodiments 15-20,
   and said infusion bag contains a diluent selected from a group of aqueous media: Ringer's lactate solution, water, saline solution, 5% dextrose solution, water for injection, and wherein
   said breakable container is placed directly inside said infusion bag suitably to allow said reconstitutable solid composition to be reconstituted upon addition of one of the above mentioned diluents by breaking said breakable container directly inside diluent in said infusion bag.
49. Medicament for use in a method of treatment or prophylaxis of bacterial infections caused by Gram-negative infections, comprising a lyophilized powder with 500 mg of a compound of formula (I) as defined in claim 1 in a 30 mL vial.
50. Medicament according to embodiment 47, wherein said medicament is further characterized by 6.5 mg/mL solved compound according to formula (I), a pH of 4.0-4.2, and 290-400 mOsmol/L upon reconstitution with an injectable reconstitution medium.
51. A process for the preparation of a formulation as defined in the embodiments 1-14, said process comprising the steps of:
   i) providing a means for mixing, preferably a mixing tank,
   ii) maintaining the bulk solution temperature at approx. 50° C. by heating means, preferably by using a heat mixing jacket,
   iii) adding approx. 60% w/v water for injection, preferably add hot 60% w/v water for injection at approx. 60° C.,
   iv) maintaining bulk solution temperature at a range of 48-55° C., preferably 49-52° C., most preferred at 50° C., whereby 50° C. is the target temperature,
   v) adding an organic and/or an inorganic acid in accordance with the invention and mix the solution, preferably mix at least for 3 minutes, more preferred at least for 4 minutes, most preferred at least for 5 minutes until dissolved,
   vi) adding a modified cyclodextrin in accordance with the invention and mix the solution, preferably mix at least for 20 minutes, more preferred at least for 25 minutes, most preferred at least for 30 minutes until dissolved,
   vii) adding a compound of the formulae (I) to (VII) as API in accordance with the invention and ensure that bulk solution temperature is at a range of 48-55° C., preferably 49-52° C., most preferred at 50° C., whereby 50° C. is the target temperature,
   viii) mixing the solution obtained under step vii) until visual dissolution is observed, and fill up to 100% bulk volume using water for injection at room temperature, thereby maintaining bulk solution at 25-35° C., preferably at 29-35° C., most preferred at 34-35° C., whereby 34-35° C. is the target temperature,
   ix) optionally take in-process sample(s) to monitor pH or for using other assays
   x) setting up a particulate reduction filter, preferably a 0.45 am particulate reduction filter, on mixing means, preferably on mixing tank
   xi) ensuring transfer line temperature is at 25-35° C., preferably at 29-35° C., most preferred at 34-35° C., whereby 34-35° C. is the target temperature,
   xii) transferring the product of step xi) immediately to filling room, as soon as bulk solution reached a temperature at 34-35° C. as target temperature,
   xiii) filtering bulk solution of step xii) through a suitable filter, preferably a 0.2 μm filter, more preferably through two 0.2 μm filter, whereby even more preferably said filter is a Polyvinylidene difluoride membrane (PVDF)
   xiv) optionally perform offline filter testing
   xv) filling bulk solution.
52. A process for the preparation of a solid composition as defined in the embodiments 15-20, said process comprising the steps of:
   xvi) lyophilizing the product obtained under step xv) of embodiment 49, and
   xvii) optionally decontaminating the lyophilized product obtained under step xvi.

With the above context of the embodiment 52, in another aspect of the invention said solid composition is a reconstitutable solid composition.
53. A process for the preparation of an aqueous injectable solution as defined in any of the embodiments 32-35, comprising the steps of:
   xviii) the lyophilisate obtained in step xvi) and optionally step xvii) of embodiment 50 is reconstituted with a suitable medium comprising water for injection, NaCl solution, dextrose solution, and Ringer's lactate solution, followed by
   xix) adding phosphate buffer/saline mixture solution for pH adjustment, so to obtain a final aqueous injectable solution for use in parenteral administration with a pH value of 4.0 to 4.5 and an osmolality of 290 to 450 mOSM/kg.
54. A solid composition as defined in any of the embodiments 15 to 20, wherein said solid composition is further formulated as oral dosage form.
55. The solid composition of embodiment 52, wherein said oral dosage forms are selected from a group comprising tablets and capsules.
56. The solid composition of embodiment 52 or 53, for use in oral administration.
57. The solid composition of any of embodiments 15-20, wherein said solid composition has been prepared from a sterile liquid formulation according to any of the embodiments 1-14 by spray-drying, freeze-drying, spray-freeze-drying, antisolvent precipitation, solvent evaporation, or by a process utilizing a supercritical or near supercritical fluid.

With the above context of the embodiments 54 to 57, in another aspect of the invention said solid composition is a reconstitutable solid composition.

In accordance with the present invention in another specific embodiment, a reconstitutable solid composition is lyophilized powder with 500 mg of a compound of formula (I) as API in a 30 mL vial. Upon reconstitution with suitable injectable reconstitution medium, the product will have 6.5 mg/mL drug content, a pH 4-4.2 and 290-400 mOsmol/L for i.v. infusion.

Other features, advantages and embodiments of the invention will become apparent to those skilled in the art by the following examples and figures, without being limited thereto.

The data provided below indicate that the API-modified cyclodextrin formulations of the invention provides for improved solubility and stability of API relative to other cyclodextrins regardless of the pH of the medium, or the charge state of the comparator cyclodextrin. Accordingly, the present invention provides an improved method of solubilizing and stabilizing API comprising the steps of including modified cyclodextrin and organic acid and/or inorganic acid in a parenteral formulation comprising API.

### Oral Administration Route

As mentioned above, in preferred aspects and embodiments of the invention, the pharmaceutical formulations of the invention will be in the form of an aqueous parenteral or injectable formulation. However, the pharmaceutical formulations of the invention may be in other dosage forms such as oral forms; for example in the form of tablets and capsules.

Thus, the reconstitutable solid compositions comprising the modified cyclodextrin complexes or the physical mixtures of the invention may also be compressed into a tablet or may be filled into capsules.

As discussed above, it is one aspect of the invention that the provided formulations improve the stability of the compounds (I) to (VII) as API.

Chemical stability is crucial for a pharmaceutical agent to maintain its activity also in forms of applicable dosage forms such as a tablet or capsule for oral use. The one skilled in the art is aware that chemical stability of an API is inter alia depending on the composition of the formulation itself, its mixture, its method of manufacture and by the storage conditions itself.

In the following, for the oral administration forms of the invention, some parameters may differ from the above mentioned formulations and compositions intended for parenteral, preferably i.v. administration. However, a person skilled in the art knows such variations when formulating oral dosage forms.

Thus, a person skilled in the art understands that the following aspects are merely preferred aspects; however, the invention shall not be limited to such specific aspects. In addition to the compounds (I) to (VII) as API, the solid pharmaceutical formulations for oral dosage forms of the present invention contain one or more pharmaceutically acceptable ingredient(s) referred to as excipients. Common excipients include inter alia fillers, diluents, binders, lubricants, glidants, disintegrants, solvents, film formers, plasticizers, pigments, and antioxidant agents. All excipients as part of the present invention are either synthetic or plant origin, they are not derived from animal or human origin.

All the listed excipients that are potentially used in the manufacture of the herein provided solid pharmaceutical formulations for oral dosage forms of the compounds (I) to (VII) as API are well known and widely used in the manufacture of pharmaceutical dosage forms (e.g. compressed tablets or capsules) using conventional pharmaceutical processes including granulation and compaction.

Thus, in another aspect of the present invention the solid pharmaceutical formulations for the use in oral dosage forms comprise one or more excipient(s) or a combination thereof selected from the group comprising microcrystalline cellulose, copovidone, croscarmellose sodium, colloidal anhydrous silica, magnesium stearate, povidone (also known as polyvinyl pyrrolidone, polyvidone or PVP), lactose, sucrose, mannitol, starch (including pregelatinised starch), talc, hydroxylpropyl cellulose, hydroxyl propyl methylcellulose (also known as hypromellose or HPMC), sodium starch glycolate, calcium hydrogenphosphate dihydrate (also known as dibasic calcium phosphate), triethyl citrate, methacrylic acid-methyl methacrylate copolymers, polyvinyl alcohol, magnesium stearate, macrogol, poly(vinylalcohol) grafted copolymer, polyvinyl acetate, methacrylic acid/ethyl acrylate copolymers.

In another aspect of the invention at least one of the compounds (I) to (VII) as API are contained in the solid pharmaceutical formulations for oral administration in the amount of 5 to 400 mg, preferably in the amount of 10 to 300 mg, more preferably in the amount of 120 to 280 mg, most preferred in the amount of 180 to 240 mg.

In another aspect, subject matter of the invention are film-coated tablets containing at least one of the compounds (I) to (VII) as API in different dose strengths, i.e. 5 mg, or 20 mg, or 30 mg, or 60 mg, or 120 mg, or 240 mg, or >240 mg of said APIs. Said distinct dose strengths should be not understood as limiting dose strengths. Any other dose strength reasonably administrable to a subject is also comprised by the scope of the present invention.

### EXAMPLES

### Example 1—Preparation of the Formulation Solutions on Lab Scale

For the SBE-β-CD/CA formulations in accordance with the invention, 9 different exemplary formulations were tested as outlined below. These formulations were high, medium, or low level of each of the two excipients SBE-β-CD and CA. The nominal formulation is medium level of both SBE-β-CD and CA. High or low level of SBE-β-CD is defined as plus or minus, respectively, of 40% of its nominal concentration. High or low level of citric acid is defined as plus or minus 1.5% CA, respectively, of 3.5% of its nominal concentration.

Exemplary SBE-β-CD/CA placebo formulation solutions were prepared by adding a compound according to formula (I) as API to said solutions directly, followed by subsequent warming of the solutions in a 50° C. water bath, so to dissolve API while shaking.

Said placebo solutions were prepared at 9 different combinations, abbreviated as LL, LM, LH, ML, MM, MH, HL, HM and HH as further set out below in Table 1, and filtered through 0.2 μm PTFE syringe filters.

The formulation solutions, duplicate of each, were prepared as listed in Table 2 (see below), while using proper selection of placebo solution. The formulation solutions were filtered through 0.2 μm PTFE syringe filters. After removing samples for initial testing, the solutions were split into 2 glass vials, stored at 5° C. and room temperature, for subsequent stability testing.

### Description of Tests

Visual appearance, pH and potency of the formulations were tested at the initial point in time (time 0; i.e. the point in time at completion of the formulation) and time points afterwards up to 4 days. Visual appearance was performed under day light conditions by eyes. Precipitation was determined when observed in a thin layer material stuck to the bottom of the vial.

pH was tested at the initial time (time 0), after 24 hours (1 day) and a the end; i.e. after 4 days. Small amount of sample was removed to record pH, so to avoid crystallization in the formulation.

A conventional HPLC method with UV detection at 260 nm was used to determine the potency and impurities of the formulations. Samples for HPLC analysis were prepared in two-step dilutions. First, the formulation was diluted to 1 mg/mL in ACN/DMSO (60/40 v/v) and stored at −20° C. The above 1 mg/mL solution was further diluted to a final analyte concentration of 20 μg/mL in 0.01% formic acid.

Crystallized and precipitated formulations were warmed at 50° C. to dissolve before sampling. Since reference standard is not available for this method, API was prepared in the same way and injected as standard for system suitability evaluation. All the sample injections were bracketed with 5 injections of API standard. Percent-RSD of API standard brackets was in the range of 0.0% to 0.3%, which indicated that the used HPLC method is accurate. Percent-Peak area of API (i.e. a compound of formula (I)) over total peaks was used to represent API potency, assuming major impurities are resolved in this method and they have the same response factors as the API main peak.

TABLE 1
DOE matrix for SBE-β-CD
(Captisol ®)/CA placebo formulation solutions:

| | Captisol | Citric Acid | | | |
|---|---|---|---|---|---|
| | (mg/mL) | (%) | | | |
| MM | 100 | 3.5 | HL | HM | HH |
| HH | 140 | 5.0 | ML | MM | MH |
| HM | 140 | 3.5 | LL | LM | LH |
| HL | 140 | 2 | | | |
| MH | 100 | 5 | | | |
| ML | 100 | 2 | | | |
| LH | 60 | 5 | | | |
| LM | 60 | 3.5 | | | |
| LL | 60 | 2 | | | |

M: medium level; L: low level; H: high level

TABLE 2
SBE-β-CD (Captisol ®)/CA formulation of API (i.e. compound (i)):

| Placebo solution | 10 mL |
|---|---|
| API | 368 mg (corresponds to 320 mg of pure API taking |
| (compound (I)) | into account correction factor of 1.15) |

Results of the SBE-β-CD/CA formulation solutions on lab scale

All tested SBE-β-CD/CA formulation solutions had similar appearance (clear, yellowish solution), only LL formulation setup with 6% SBE-β-CD+2% CA precipitated at 5° C. (see the table in FIG. 1).

Moreover, it has been surprisingly found that pH is mainly affected by the concentration of CA applied, whereas SBE-β-CD showed no effect on pH.

For instance, a CA concentration of 2% shifted the pH to a range 2.9-3.0. A CA concentration of 3.5% shifted the pH to a range 2.6-2.7. And a CA concentration of 5% shifted the pH to a range of 2.4-2.6 (see FIG. 2).

The SBE-β-CD/CA formulation solutions had only one major degradant that increased significantly through time course (see FIG. 3 and HPLC of FIG. 4).

Moreover, it was found that temperatur is a key factor affecting the API's stability (here exemplarily shown for a compound according to formula (I) in the plot of FIG. 5).

API stability showed that the 5° C. stored samples (blue symbols in FIG. 5) are in the trends well separated from the room temperature stored samples (red symbols in FIG. 5). The data are fitted into the trend line of y=−ax+100. The slope a, representing the API degradation rate in percentage per day, is listed in FIG. 6).

Besides the temperature, the influence from the excipients to the API stability is also clear in the SBE-β-CD/CA formulation solutions. At the same concentration of SBE-β-CD and storage temperature, the API degradation rate has a positive correlation with CA concentration.

For example, the formulations ML, MM and MH at room temperature showed a degradation rate of 3.39% per day, 3.79% per day and 4.17% per day, respectively. On the other hand, SBE-β-CD seems to protect API from degradation that at the same CA and storage condition, the API degradation rate shows a negative correlation with the level of SBE-β-CD.

For example, degradation rates of formulations LL, ML and HL at room temperature are 3.73% per day, 3.39% per day and 3.16% per day, respectively.

Combining all the influences recognized from the excipients, storage, and temperature conditions applied, it has been surprisingly found that the formulation HL (140 mg/mL SBE-3-CD/2% CA) at 5° C. showed the best API stability at a degradation rate of only 0.74% per day.

With this context, it could be found that the higher the CA concentration is, the more degradation can be observed (ML 3.39%, MM 3.79%, ML 4.17% per day), and that SBE-3-CD in specific concentration ranges protects the API from degradation (LL 3.73%, ML 3.39%, HL 3.16% per day).

### Example 2—Scale Stability

In this study on stability of the compound (I) as API in the formulations of the invention, exemplary API 50 mL batches in 20% SBE-β-CD and 1% CA have been tested for photostability and storage stability up to 12 months.

Therefore, experiments have been conducted to evaluate the critical quality attributes of the drug product (i.e. a compound of the formula (I)) at different storage conditions.

Example 2 summarizes the testing results of photostability and stability study at the following points in time: time zero (initial), 1 month, 2 months, 3 months, 6 months, 9 months and 12 months.

### Batch Production

A 3 L batch (# WO 2015-0213) was manufactured on Jun. 15, 2015 at Lake Forest. The formulation was compounded and filtered in the lab (see the formulations in Table 3); filling and lyophilization was performed in the pilot plant. The target fill volume was 15.6 mL per vial. The vials were half stoppered and subjected to freeze drying in Edwards Lyoflex 0.4 Lyophilizer under a product temperature driven cycle. The lyophilization was finished on Jun. 19, 2015. 141 amber vials and 19 clear vials were obtained.

TABLE 3
Formulation of 3 L batch (# WO 2015-0213)

| | | | Actual Amount |
|---|---|---|---|
| Ingredient | Concentration | Amount per Liter | in the Batch |
| API (compound (I)) | 32 mg/mL | 35.2 g * | 105.6 g |
| CA | 1% | 10 g | 30.0 g |
| SBE-β-CD | 20% | 200 g | 600.0 g |

* The amount of API per liter is corrected with a factor of 1.10 for Batch # WO 2015-0213. The correction factor was calculated from water content, UPLC impurities and sulfated ash based on the API Certificate of Analysis (see FIG. 19).

### Study Design

The stability storage of the tested formulations of API (compound (I)) was initiated on Jun. 25, 2015 according to the Table 4. The test methods and tentative specifications are summarized in Table 5.

TABLE 4
Stability storage conditions and test intervals

| Storage | | Storage | Month | | | | | | |
|---|---|---|---|---|---|---|---|---|---|
| Condition | Orientation | Location | 0 | 1 | 2 | 3 | 6 | 9 | 12 |
| Subzero | Upright | Lake | X | X | X | X | X | X | X |
| −25 to | Inverted | Forest | | | | | X | | X |
| −10° C. | | | | | | | | | |
| Real Time | Upright | Lake | | X | X | X | X | X | X |
| 2 to 8° C. | Inverted | Forest | | | | | X | | X |
| Accelerated | Upright | Pleasant | | X | X | X | X | X | X |
| 25° C./ | Inverted | Prairie | | | | | X | | X |
| 60% RH | | | | | | | | | |

TABLE 5
Test methods and tentative specifications

| Test | Tentative Specification | Amount |
|---|---|---|
| Appearance (lyophilised) | Record results | NA* |
| Water Content by Karl Fischer | Record results | 3 vials |
| Reconstitution Time | Record results | NA** |
| Reconstituted with 15 mL water | | |
| Appearance of solution | Clear solution. Free of visible | NA** |
| Reconstituted to 50 mL water | particles | |
| pH | Record results | NA** |
| Reconstituted to 50 mL water | | |
| Id by UPLC | RT within RT standard ± 0.4 min | 1 Vial |
| Purity by UPLC | 93.0%-107.0% (release) | |
| AIC214006 % Peak Area | 92.0%-108.0% (shelf-life) | |
| Assay by UPLC | 93.0%-107.0% (release) | |
| AIC214006, % Label Claim | 92.0%-108.0% (shelf-life) | |
| Impurities by UPLC | A. NMT 3.0% (release) | |
| A. Ring opened API | NMT 5.0% (shelf-life) | |
| B. Individual Unspecified | B. NMT 0.6% | |
| Impurities | C. NMT 5.0% (release) | |
| C. Total Impurities | NMT 8.0% (shelf-life) | |

*Tests performed on the same vials as water content
**Tests performed on the same vial as UPLC

### Photostability

Photostability of the API formulation solutions was evaluated in 50 mL clear vials. The 10 clear vials made in the batch were used in the study; 5 were wrapped in aluminum foil and were used as control. Both controls and the samples were subjected to the photostability parameters specified in ICH Q1B guideline at controlled temperature at 25° C. All vials were exposed to an overall illumination of 1.2 million lux hours of visible light and 200 watt hours/m² of near UV radiation in a Caron 6540 Photostability Chamber, using D65 lamps according to ICH Q1B, Option 2. After exposure, samples were maintained at 2 to 8° C. until analysis.

### Results Photostability

In general, the light exposed product was bright yellow compared to off-white of the control sample. In addition to the appearance, the exposed product had lower purity and potency along with higher impurities when compared to the control.

Results from initial and end point testing are listed in FIG. 7. Results of control samples are similar to those of the initial samples, both met proposed specifications. The light exposed product was bright yellow compared to off-white of the control sample (see FIG. 7). In addition to the appearance, the exposed product had lower purity and potency along with higher impurities when compared to the control (FIG. 7).

FIG. 8 shows a chromatogram overlay of the control and light exposed samples. Three impurities at relative retention time (RRT) 0.22, 0.26 and 1.28 increased significantly in the exposed sample compared to the control. The impurity at RRT 1.28 was 1.0%, exceeding the proposed specification of NMT 0.6%.

In conclusion, based on the analytical results of the photostability experiments the tested API formulation solutions (i.e. with a compound of formula (I)) are susceptible to light exposure according to current ICH Q1B guideline. Therefore, the final drug product will be stored protected from light.

### Stability Results

Stability testing has been conducted through 12 months as shown in the Tables 4 and 5 above. Thus, three different storage conditions in upright and inverted orientation were applied as follows:
Real time under 2-8° C. for 12 months
Accelerated conditions of 25° C./60% RH for 6 months
Subzero conditions with −25-−10° C. for 12 months.

Testing results of the initial and 1 month time point up until 12 months are summarized below.

No significant differences of the results from the samples stored at 25° C./60% RH in inverted condition could be obtained.

### Appearance of Lyophilisate

The lyophilised API (compound (I)) solid composition is an off-white friable cake with sheen finish and cracks on the surface. The product is uniform in color. There are no changes in appearance up to 12 months at any storage condition (see FIG. 9).

### Water Content

Water content of the lyophilised API (compound (I)) solid composition was determined by coulometric Karl Fischer method based on direct addition. Results are summarized in FIG. 10. There are no significant changes in water content up to 12 months for any storage condition.

### Reconstitution Time

Reconstitution was conducted by adding 15 mL of water to one product vial containing the solid compositions of the invention with a compound of formula (I) as API. The reconstitution time is determined by when all freeze dried product is dissolved into clear solution. Results are summarized in FIG. 11. There are no significant changes in reconstitution time up to 12 months for any storage condition.

### Appearance of Solution

All the samples have appearance of reconstituted solution as being conform to proposed specification of clear solution, free of visible particulates. Results are summarized in FIG. 12.

pH

The pH of the reconstituted solution at 50 mL was tested. Results are summarized in FIG. 13. There are no changes in pH results of the reconstituted solution up to 12 months at any storage condition.

### Id by UPLC

All the samples were subjected to Id testing by UPLC. All samples are conform to proposed specification of retention time (RT)±0.4 min. of standard. Results are summarized in FIG. 14.

Purity by UPLC—% Peak Area of API (i.e. Compound (I))

Results are summarized in FIG. 15. The product purity, i.e. the purity of a pharmaceutical composition in accordance with the invention containing a compound of formula (I) as API upon reconstitution, after 1 month at the real time and subzero conditions are similar to that of initial testing result. The accelerated product had a 0.7% purity loss compared to the initial results. At 2 months and 3 months, samples stored at the real time and subzero conditions have similar purity to that of initial samples. The accelerated product has progressive purity loss across time intervals. At 9 months, samples stored at 2-8° C. and subzero conditions showed no significant differences in terms of purity (% peak area) comparing to the results from previous time points. Samples stored at the accelerated condition had significant purity loss compared to that of the initial testing result. At 12 months, samples stored at 2-8° C. and subzero condition showed no significant difference in purity (% peak area) compared to the results from previous time points. Samples stored at the accelerated condition had significant purity loss compared to that of the initial testing result, but no difference when compared to the 9 month sample.

### Impurities by UPLC—Ring Open API (i.e. a Compound of Formula (I))

Results are summarized in FIG. 16. There is no significant change in Ring open API impurity after 1 month at any storage condition. At 2 months and 3 months, the Ring open API impurity results are similar between all three storage conditions. At 6 months, the Ring open API impurity results are similar between all three storage conditions and increased compared to previous time points. At 9 months, the Ring open API impurity results are similar between all storage conditions. At 12 months, the Ring open API impurity results are similar between all three storage conditions no change compared to 9 month time point could be observed.

### Impurities by UPLC—Single Largest Unspecified Impurity

More than 10 unspecified impurities could be determined by UPLC method. The single largest unspecified impurity after 1 month at the real time and subzero conditions is similar to that of initial testing result. Accelerated product has higher single largest unspecified impurity when compared to the initial results. Results are summarized in FIG. 17. At 6 months, samples stored at subzero conditions have the same level of single largest unspecified impurity compared to previous samples; samples stored at the real time conditions have slightly higher single largest unspecified impurity than at 3 months; the accelerated product has progressive increase in single largest unspecified impurity across time intervals. There is no difference between samples stored at inverted or upright orientations. At 12 months, there is no change in the single largest impurity at any of the conditions tested.

### Impurities by UPLC—Total Impurities

Results are summarized in FIG. 18. The total impurities after 1 month at the real time and subzero conditions are similar to that of initial testing result. The accelerated product has higher total impurities compared to the initial results. At 6 months, samples stored at all three conditions have higher total impurities than at 3 months. Samples stored at inverted or upright orientations have similar total impurities. At 9 months, samples stored at real time and subzero conditions did not show significant changes from previous results. The sample stored at accelerated condition has slight increase in total impurities compared to the 6 months results. At 12 months, samples stored at real time, accelerated and subzero conditions did not show significant change from the 9 month results.

### Example 3—Solubility of a Compound of Formula (I) as API in SBE-β-CD

The solubility of a compound of formula (I) as API is highly pH dependent as shown in FIG. 23.

The corresponding phase-solubility profile appears to be linear, i.e. of A_{L}-Type (in the concentration range of 0-20% SBE-β-CD. Deviations could perhaps be due to pH fluctuations (see FIG. 24).

The A_{L}-type phase-solubility profile as depicted in FIG. 24 indicates formation of compound (I)-SBE-β-CD 1:1 complex, that one compound (I) molecule forms a complex with one SBE-β-CD molecule. Accordingly, the stability constant (K_{1:1}) of the complex can be estimated from the intrinsic solubility (i.e., the solubility when no SBE-β-CD is present or S₀) and the slope of the linear profile by the following formula:

$${K}_{1 \text{:} 1} = \frac{Slope}{{S}_{0} \cdot ( 1 - Slope )}$$

The solubility profile of the API of a compound of formula (I) was measured at two different pH values (see FIG. 25):

$$At pH 4.0 \text{:} {K}_{1 \text{:} 1} \approx \frac{0.331}{0.015 \cdot ( 1 - 0.331 )} = 33 {M}^{- 1}$$

$$At pH 7.4 \text{:} {K}_{1 \text{:} 1} \approx \frac{0.411}{0.0015 \cdot ( 1 - 0.411 )} = 465 {M}^{- 1}$$

The MW of compound (I) (668.7 Da) and of 20% (w/v) SBE-β-CD (2163 Da) is 0.0936 mole/liter.

### Objective

To determine the solubility of a compound (I) in selected solvent systems in accordance with the invention, UPLC was used, and physicochemical properties such as pH and physical appearance were monitored.

Materials and equipments
a) Compound (I)
b) Hydroxypropyl-β-cyclodextrin (HP-β-CD)
c) citric acid
d) Polyethylene glycol 400 (PEG 400)
e) Acetonitrile, HPLC grade
f) Dimethyl sulfoxide
g) Formic acid
h) Purified water
i) Ammonium formate
j) UPLC system details:

Waters Acquity UPLC with PDA detector
k) Column: Phenomenex Kinetex XB-C18, 100×2.1 m; column packing particle size 2.6 μm.
l) pH meter:
m) Magnetic stirrer

### Mobile Phase and Solution Preparation

### a) Mobile Phase

Mobile phase A: 950 mL water+50 mL 200 mM ammonium formate

Mobile phase B: 900 mL acetonitrile+50 mL water+50 mL 200 mM ammonium formate.

### b) Stock Diluent

Stock diluent is a mixture of acetonitrile and DMSO (60%+40%). 100 mL of stock diluent were prepared by mixing accurately measured volumes of acetonitrile (60 mL) and DMSO (40 mL).

### c) Analysis Diluent

Analysis diluent is a 0.01% formic acid solution in water. 100 mL of analysis diluent were prepared by adding 0.01 mL of formic acid into 50 mL of water in a 100 mL capacity volumetric flask and then the volume of this solution was made up to 100 mL with water.

### d) Preparation of Standard Solution

20 mg of compound (I) were accurately weighed in a 20 mL volumetric flask and completely dissolved using the stock diluent and the volume was filled up with the same. This is a stock solution.

The above stock solution is further diluted with 0.01% formic acid to get a concentration of 10 μg/mL.

e) Hydroxypropyl-β-Cyclodextrin Solution (30% w/v)

In a 25 mL capacity volumetric flask accurately weighed 7.5 mg of HP-β-CD were added and dissolved in sufficient amount of water, final volume was adjusted to 25 mL using water.

f) Hydroxypropyl-β-Cyclodextrin (30% w/v) Solution Containing 2% CA

In a 25 mL capacity volumetric flask accurately weighed 7.5 mg of HP-β-CD were added and dissolved in sufficient amount of water, final volume was adjusted to 25 mL using water. To this solution 0.5 mg of CA were added and vortexed for few seconds to obtain a clear solution.

### g) Solvent Mixture of PEG400 (40%)+2% CA (60%)

In 50 mL capacity volumetric flask accurately weighed 1 mg of CA was added and dissolved in sufficient amount of water. Final volume was adjusted to 50 mL with water.

Solvent mixture was prepared by mixing 15 mL of 2% CA with 10 mL of PEG 400 in a 50 mL capacity glass bottle.

### h) Preparation of Test Sample

0.5 mL of test solution was diluted to 5 mL with stock diluent. This solution was then subsequently diluted with the analysis diluent.

### UPLC Method

A standard solution of compound (I) (10 μg/mL) and a blank, 0.01% formic acid solution were analysed on a UPLC system using the following method:

### Gradient:

| Time (min.) | Flow (mL/min.) | Mobile Phase A (%) | Mobile Phase B (%) |
|---|---|---|---|
| 0.00 | 0.2 | 99 | 1 |
| 1 | 0.2 | 99 | 1 |
| 15 | 0.2 | 88 | 12 |
| 20 | 0.2 | 75 | 25 |
| 20.5 | 0.2 | 5 | 95 |
| 23 | 0.2 | 5 | 95 |
| 23.1 | 0.2 | 99 | 1 |
| 28 | 0.2 | 99 | 1 |

Injection volume: 10 μL
Detection wavelength: 260 nm

### Solubility Experiment

The solubility of compound (I) in solution was determined at 25° C. separately in following solvent systems at three time points (at 2 hours, at 6 hours and at 24 hours) in duplicates.

1) Hydroxypropyl-β-cyclodextrin solution (30% w/v)
2) Hydroxypropyl-β-cyclodextrin (30% w/v) solution containing 2% CA
3) Solvent mixture of PEG400 (40%)+2% CA (60%)

A common procedure was followed to determine the solubility of compound (I) in all above listed solvents.

### Procedure:

To the vial for each time point 3 mL of individual solvent were added followed by addition of the weighed amount of compound (I) (100 mg). This system was kept for stirring on a magnetic stirrer at 700 rpm. At each time point, the solution was filtered through a 0.22 am syringe filter and analysed for the content of compound (I) using UPLC after suitable dilutions.

During solubility experiments all vials were covered with aluminium foil.

### Results Solubility Testing

The results of solubility testing are summarised in FIGS. 26-27.

The solubility data of FIGS. 26-27 demonstrate that with 30% HP-β-CD as sole excipient, solubility of compound (I) is 2.7 mg/ml. When 2% CA is added as additional excipient to 30% HP-β-CD, solubility value of compound (I) is increased significantly up to 16.18 mg/ml. This demonstrates one of the key aspects of the invention, namely the solubility enhancing effect of a zwitterionic compound such as compound (I) in presence of a modified cyclodextrin in specific ranges combined with an organic acid such as citric acid, in specific ranges.

### Example 4—Exemplary Parenteral Solution

A parenteral solution (i.e. an i.v. injectable solution) in accordance with the invention contains 5 mg/ml (0.007477 M) of compound (I) as API and 31 mg/ml (0.01444 M) of SBE-β-CD, at pH 4.0-4.2 with an osmolarity of 290-400 mOsmol/liter. The fraction of compound (I) bound to SBE-β-CD in the aqueous injectable parenteral solution is calculated by the following formula:

$${f}_{bound} = \frac{[ D / CD ]}{[ D ] + [ D / CD ]} = \frac{{K}_{1 \text{:} 1} \cdot [ SBE β CD ]}{1 + {K}_{1 \text{:} 1} \cdot [ SBE β CD ]} = \frac{33 \cdot 0.01444}{1 + 33 \cdot 0.01444} = 0.70 or 70 \%$$

On average, mice have 79 ml of blood per kg of body weight (see https://en.wikipedia.org/wiki/Blood_volume; Oct. 3, 2016). If the blood plasma is 55% of the total blood volume then mice have around 43 ml of plasma per kg of body weight. The dose was 25 mg/kg (0.037 mmoles/kg) of compound (I) and 156.5 mg/kg (0.0.0724 mmoles/kg) of SBE-β-CD. After mixing with the blood plasma the initial plasma concentration should be 8.6.10⁻⁴ M of compound (I) and 1.7.10⁻³ M of SBE-β-CD. Ignoring drug plasma protein binding and drug tissue binding the fraction of compound (I) bound to SBE-β-CD in the blood plasma (at pH 7.4) is according to the below formula:

$${f}_{bound} = \frac{[ D / CD ]}{[ D ] + [ D / CD ]} = \frac{{K}_{1 \text{:} 1} \cdot [ SBE β CD ]}{1 + {K}_{1 \text{:} 1} \cdot [ SBE β CD ]} = \frac{465 \cdot 0.0017}{1 + 465 \cdot 0.0017} = 0.44 or 44 \%$$

However, the fraction of compound (I) bound to plasma protein in mice (fₚ) is 0.221 and if the plasma protein concentration is 6·10⁻⁴ M then we get:

$${K}_{P} = \frac{{f}_{P}}{[ P ] \cdot ( 1 - {f}_{P} )} = \frac{0.221}{6 \cdot {10}^{- 4} \cdot ( 1 - 0.221 )} = 473 {M}^{- 1} or \frac{{K}_{p}}{{K}_{1 \text{:} 1}} \approx 1$$

The compound (I) has as much affinity for the plasma protein than for SBE-β-CD and, thus, only about 22% of compound (I) is bound to SBE-β-CD after i.v. administration at time zero, about 80% of compound (I) is either free or bound to plasma proteins. But then again the fact would be ignored that most plasma proteins have more than one binding site for drugs and competitive displacement of compound (I) from SBE-β-CD and we do not account for drug tissue binding.

Cholesterol and other endogenous compounds will have some affinity to SBE-β-CD and bind to SBE-β-CD in plasma reducing compound (I) binding to SBE-β-CD. This will lower the fraction of compound (I) bound to SBE-β-CD well below 20%. The effects of cyclodextrins and modified cyclodextrins on the pharmacokinetics of drugs after parenteral administration has been reviewed and it is generally accepted that cyclodextrins and modified cyclodextrins will not affect the pharmacokinetics of drugs when the K 1:1 value is below 104 to 105 M⁻¹.

The stated above can be dervied from the following literature citations:
1. Stella V J, He Q. Cyclodextrins. Toxicologic Pathology. 2008; 36(1):30-42.
2. Kurkov S V, Loftsson T, Messner M, Madden D. Parenteral delivery of HPβCD: effects on drug-HSA binding. Aaps Pharmscitech. 2010; 11(3):1152-8.
3. Stella V J, Rao V M, Zannou E A, Zia V. Mechanisms of drug release from cyclodextrin complexes. Adv Drug Deliver Rev. 1999; 36(1):3-16.
4. Loftsson T, Moya-Ortega M D, Alvarez-Lorenzo C, Concheiro A. Pharmacokinetics of cyclodextrins and drugs after oral and parenteral administration of drug/cyclodextrin complexes. J Pharm Pharmacol. 2015; 67.
5. Kurkov S V, Madden D E, Carr D, Loftsson T. The effect of parenterally administered cyclodextrins on the pharmacokinetics of coadministered drugs. J Pharm Sci-Us. 2012; 101(12):4402-8.

### Example 5—Precipitation of Compound of Formula (I) as API Upon i.v. Injection

An exemplary parenteral pure “saline” solution at pH 4.0 contains 5 mg/ml API of compound (I) only. The compound (I) solubility at pH 4.0 is 10 mg/ml. Thus, this exemplary parenteral “saline” solution contains no modified cyclodextrin or other solubilizer, and is far from saturated with the drug.

However, upon i.v. administration the pH of the drug solution at the site of injection will almost instantaneously increase from pH 4 to 7.4. The solubility of compound (I) at pH 7.4 is 1 mg/ml or five times lower than the compound (I) concentration in the aqueous SBE-β-CD free parenteral solution in accordance with the invention. Thus, without no modified cyclodextrin or other solubilizer some compound (I) precipitation at the injection site is likely. Compound (I) precipitation could explain the lower Cp values obtained after injection of the SBE-β-CD-free parenteral solution and the slightly larger tv₂ value, as well as larger Cl_{T} and V_{d}.

It should be mentioned that drug precipitation after i.v. administration of cyclodextrin containing parenteral solutions is very uncommon while drug precipitation after parenteral administration of identical concentrations of the same drug in organic solvents (like DMSO), or upon pH adjustments, is not uncommon.

### Example 6—In Vivo Testing for Tolerability

The aim of this example was to test the local tolerance of a compound (I) containing aqueous injectable solution in accordance with the invention in female rabbits following a single intravenous infusion for 30 or 60 minutes. Each animal received 15 mL of a formulation containing 5 mg compound (I) as API per mL into the marginal vein of the right ear. Two groups of 9 female animals, each, were employed with infusion durations of 30 or 60 minutes per dose.

In addition, a placebo solution as vehicle control, was administered in the same volume and duration on the left ear of each animal. 24, 72 and 96 hours after administration respectively 3 animals per group were sacrificed and the injection sites were examined macro- and microscopically.

### Tested Compound—Compound (I)

Characteristics: lyophilisate
Appearance: Off-white to tan colour
Water content: 2.7%
Storage conditions: at <−20° C., protected from light
Stability: 6 hours at room temperature after
reconstitution; storage at +2° C. to +8° C. of
the reconstituted compound (I)
Purity: 2.0% ring open API impurity
0.2% single largest unknown impurity
3.3% total impurities

### Placebo

Characteristics: lyophilisate without a compound (I)
Appearance: white
Water content: 2.9%
Storage conditions: at <−20° C., protected from light
Purity: not applicable

### Vehicle

The vehicle is composed of water for injection (WFI) and Ringer's lactate buffer solution.

### Preparation of the In Vivo Application Solutions

The application formulations were freshly prepared on the administration day. The compound (I) and the placebo were dissolved in the vehicles to the appropriate concentrations:

### 1) Reconstitution

15 mL of WFI were added to 1 compound (I) drug product vial and shaken well. The solution volume expanded to approximately 17 mL. The final concentration was approximately 29 mg/mL.

Optionally: The reconstituted solution was filtered through a 0.2 μm PVDF for intended use.

### 2) Dilution

Withdraw 17 mL from 100 mL of lactated Ringer's solution. Then add the reconstituted solution (approx. 17 mL from step 1). Mix well. The final concentration was approximately 5 mg/mL.

Steps 1 and 2 were repeated for the reconstitution and dilution of the Placebo.

### Animals/Animal Maintenance

Species: rabbit; Strain: New Zealand White
Selection of species: the rabbit is a commonly used species for local tolerance studies.
Number and sex of animals: 18 female animals
Age (at start of administration): approx. 3 months
Body weight (at start of administration): 2.31 to 2.73 kg
Acclimatisation period: at least 20 acclimatisation days; 4 test days

### Administration

Routes of administration: single intravenous infusion into the marginal vein of the ear. Selection of routes of administration: according to clinical use via intravenous route.

The application areas were sheared and disinfected with 70% ethanol before administration. The infusion sites were marked with India ink.

The compound (I) solutions were administered to the right ear, and the placebo solutions were administered to the left ear of each animal.

### Local Reactions

Local reactions were inspected macroscopically 1 h, 2 h, 6 h, 24 h, 48 h, 72 h and 96 h after injection. The reactions were scored on the basis of DRAIZE, Appraisal of the Safety of Chemicals in Food, Drugs and Cosmetics, Association of Food and Drug Officials of the United States, Austin, Tex., 1959.

### Sacrifice and Histopathology

The animals scheduled for the respective dissection day were sacrificed with pentobarbitol injection into an ear vein (not used for infusion). All animals were subjected to gross examination including the opening of the cranial, thoracic and abdominal cavities and the examination of the major organs. Particular attention was paid to the appropriate injection sites (test and control items). All abnormalities were recorded.

Tissue abnormalities would have been preserved in 10% neutral buffered formalin. Histopathological examination was performed on treated infusion sites (compound (I) and placebo, 2 sites per animal) plus an untreated adjacent site from all animals. Tissue samples were fixed in 10% buffered formalin. Paraffin sections (3 to 5 μm) were prepared, stained with hematoxylin-eosin, and examined histologically.

### Results—In Vivo Tolerability Testing

Macroscopic changes: Macroscopic inspection of the infusion sites did not reveal any changes. Necropsy did not reveal any changes, either.

Microscopic changes: The histomorphological examination of the infusion sites did not reveal any compound (I)-related changes in any of the compound (I)-treated infusion sites compared to the vehicle control sites. All changes observed are regarded as unspecific reactions caused by the infusion procedure.

Clinical signs: No clinical signs of toxicity were observed.

Body weight and food consumption: No influence on the body weight was observed.

In conclusion, a single intravenous infusion of 15 mL of a solution containing 5 mg/mL of compound (I) did not cause any compound (I)-related histopathological changes at the infusion sites, neither after 30 minutes nor after 60 minutes infusion duration.

The compound (I) showed a very good compatibility following intravenous infusion.

### Example 7—Stopper Compatibility

During the product manufacturing, the liquid formulations of the invention could contact the rubber material of stoppers, which might cause some potential risks such as leachable impurities from the stoppers or adsorption of the API to the stoppers.

Below, the potential risks of two commercially used lyo stoppers from West Pharmaceutical Services were assessed. Thus, the stopper compatibility of the aqueous injectable solutions of the invention containing a compound (I) was tested.

### Formulation and Testing

A 50 mL lab batch formulation of 32 mg/mL compound (I) in 10% SBE-β-CD/2% CA was prepared. The batch was filtered and 5 mL were filled into each 25 mL glass vials and capped with stoppers. The vials were placed at inverted orientation and stored at room temperature. As a control, the same amount of the formulation from the same batch was filled and placed upright without touching the stopper. The fill volume of 5 mL per vial is the minimum amount that ensured the stoppers immersed in the liquid formulation at the inverted orientation through the time course.

In addition, the smaller volume than the full fill of 15.6 mL can amplify the potential changes of the formulation during the stopper contact. Storage was at room temperature to mimic the clinical conditions when the lyophilized product is reconstituted back to liquid.

It was previously found that the 10% SBE-β-CD/2% CA formulation provides less protection of API than the 20% SBE-β-CD/1% CA formulation.

Therefore, the usage of the 10% SBE-β-CD/2% CA formulation in this example covers the 20% SBE-β-CD/2% CA formulation.

Three replicate vials were prepared for each stopper and the control. The potency and impurities of the formulation samples were tested at time 0, and then every 24 hours up to 72 hours. Since the pH of the formulation solutions is stable based on previous formulations, the pH of the solutions were only tested at 72 hours, the end of testing time course.

### Results—Stopper Compatibility

FIG. 28 lists the pH results of all the 9 samples at the end of testing time course. There is no significant difference between the control samples and stopper contacted samples.

An HPLC-UV method was used to test the compound (I) potency and impurities of the formulation for the stopper compatibility example. The API % peak area is used to represent the API potency assuming API and degradation products have similar response factor in this method.

FIG. 29 lists the potency of the tested formulation samples. There is also no significant difference between the control samples and stopper contacted samples. A corresponding plot is shown in FIG. 30.

At each time point through the course of time zero to 72 hours, there is no significant difference in API % peak area for either of the stoppers as compared to the control. Though API potency decreased in all the 9 samples, there is no significant difference in the degradation rate for the control and stoppers contacted samples (FIG. 30). The degradation rate of 3.4%-3.5% per day at room temperature is similar to observations in previous experiments.

### Conclusion—Stopper Compatibility

Based on the results, it is concluded that the West Pharmaceutical Services stoppers 4432/50 and 4405/50 do not alter pH and potency of API (i.e. tested compound (I)) in the 10% SBE-β-CD/2% CA formulation.

### Example 8—Process for the Preparation of a Lyophilized Formulation According to the Present Invention

1) 68 kg of water for injection was added to the mixing vessel:

The water for injection temperature must be maintained between 48-55° C., particularly, 50° C.

2) Subsequently, about 0.5% to 1.5% of citric acid (in this example 623.58 g of citric acid) was added via the hopper into the mixing vessel and bag was rinsed with a small amount of water for injection. It was ensured that impellor is switched on during citric acid addition. The acid is, stirred until it is dissolved and the temperature is maintained between 48-55° C. (in particular 50° C.)
3) Thereafter, 10% to 30%, in the present case 12470.6 g of the Captisol was added via the hopper into the mixing vessel and bag was rinsed with a small amount of water for injection. It was ensured that impellor is on during Captisol addition, stired until dissolved. It was mixed for a minimum of 30 minutes after the addition of the Captisol and until visual dissolution was observed. The temperature is maintained between 48-55° C. (in particular 50° C.)
4) Finally, the active ingredient according to the present invention (2.650 Kg) was provided in pre-dispensed Hicoflex bag. Hicoflex bag was attached to the hicoflex adapter on the mixing vessel TAMX039 and it was confirmed that 2″ Saunders valve is closed:
It was ensured that impellor is on during API addition, stirred until dissolved.
It was ensured that bulk solution temperature was between 48-55° C. (in particular 50° C.) during APT addition. After visual dissolution of API was achieved, it was ensured that bulk solution was adjusted to 25° C.-35° C. (in particular 34° C.)

### Batch Volume Adjustment:

Batch volume was adjusted with the impeller stopped using WFI for final batch weight It was ensured that bulk solution temperature is between 25-35° C. (in particular 34° C.)

### Filtration and Filling:

The solution was filtered through two 0.2μ MCY 4440DFLPH4 filters aseptically and the filtrated solution was filled in aseptically in the 50 ml amber coloured vials

Lyophilization generally is performed using loading, freezing, evacuation, and drying steps. In this example, the vials were subjected to freeze drying using a cycle of

| | | | Vacuum/ | |
|---|---|---|---|---|
| | | Temperature | Pressure | Time |
| | Step | (° C.) | (micr.) | (hh:mm) |
| 1 | Loading | 5 | | |
| 2 | Freezing | −35 | | 01:20 |
| 3 | Product Hold | −30 | | 04:00 |
| 4 | Evacuation | | 100 | |
| 5 | Product Hold | −35 | | 00:00 |
| 6 | Drying | −5 | 100 | 02:00 |
| 7 | Product Hold | −8 | | 10:00 |
| 8 | Drying | 20 | 100 | 00:50 |
| 9 | Product Hold | 17 | | 08:00 |
| 10 | Drying | 5 | 800 | 00:00 |
| 11 | Product Hold | 8 | | 72:00 |

### FIGURE DESCRIPTION

The following drawings are part of the present description and are included to further demonstrate certain aspects of the invention. The invention may be better understood by reference to one or more of these drawings in combination with the detailed description of the specific embodiments presented herein.

FIG. 1: Visual appearance of the SBE-β-CD/CA formulations tested in Example 1.

FIG. 2: pH of the SBE-β-CD/CA formulations tested in Example 1.

FIG. 3: Potency of the SBE-β-CD/CA formulations tested in Example 1.

FIG. 4: Representative chromatograms of SBE-β-CD/CA formulations tested in Example 1. Showing overlay of a compound of formula (I) as API and the tested nominal formulations. Top: full chromatograms; Bottom: zoomed chromatograms to show details.

FIG. 5: Potency plot of the SBE-β-CD/CA formulations tested in Example 1.

FIG. 6: Degradation rate of the SBE-β-CD/CA formulations tested in Example 1.

FIG. 7: Photostability testing results.

FIG. 8: Chromatogram overlay—zoomed into baseline to show impurities. FIG. 8 shows a chromatogram overlay of the control and light exposed samples. Three impurities at relative retention time (RRT) 0.22, 0.26 and 1.28 increased significantly in the exposed sample compared to the control. The impurity at RRT 1.28 was 1.0%, exceeded the proposed specification of NMT 0.6%.

FIG. 9: Stability results of testing exemplary solid compositions with a compound of formula (I) as API up to 12 months under different storage conditions. Appearance of lyophilisate (record results).

FIG. 10: Water content results by coulometric Karl Fischer method applied to the tested exemplary solid compositions with a compound of formula (I) as API up to 12 months (record results).

FIG. 11: Reconstitution time results of the tested exemplary solid compositions with a compound of formula (I) as API up to 12 months (record results).

FIG. 12: Appearance of reconstituted solution, i.e. a pharmaceutical composition in accordance with the invention comprising a compound of formula (I) as API (clear solution, free of visible particulates).

FIG. 13: pH of reconstituted solution, i.e. a pharmaceutical composition in accordance with the invention comprising a compound of formula (I) as API (record results).

FIG. 14: Id by UPLC (RT±0.4 min of standard).

FIG. 15: Purity by UPLC—% Peak Area (93.0%-107.0% at release; 92.0%-108.0% shelf-life)

FIG. 16: Impurities—Ring open API (i.e. a compound of formula (I); % Peak Area (NMT 3.0% at release; NMT 5.0% shelf-life).

FIG. 17: Impurities—single largest unspecified impurities % peak area (NMT 0.6%).

FIG. 18: Total impurities % peak area (NMT 5.0% at release; NMT 8.0% shelf-life).

FIG. 19: Certificate of analysis for batch IC1500002A of an exemplary reconstitutable solid composition containing a compound of formula (I) as API.

FIG. 20: Schematic workflow of the manufacturing process to obtain a reconstitutable solid composition in accordance with the invention.

FIG. 21: Exemplary mg/mL amounts of ingredients of a formulation of the invention prior to lyophilisation, as lyophilisate and upon reconstitution.

FIG. 22: Exemplary amounts on a %-basis of ingredients of a formulation of the invention prior to lyophilisation, as lyophilisate and upon reconstitution.

FIG. 23: pH solubility profile in pure water at 25° C.

FIG. 24: Phase solubility profile at pH 4.

FIG. 25: Solubility of a compound (I) in presence and absence of SBE-β-CD at pH 4.0 and pH 7.4.

FIG. 26: Solubility of compound (I) in different solvent systems.

FIG. 27: pH measurements during solubility testing.

FIG. 28: pH of the 10% SBE-β-CD/2% CA formulation in the upright control vials and inverted stopper vials at 72 hours.

FIG. 29: Potency of the 10% SBE-β-CD/2% CA formulation samples.

FIG. 30: Plot of compound (I) % peak area over time.

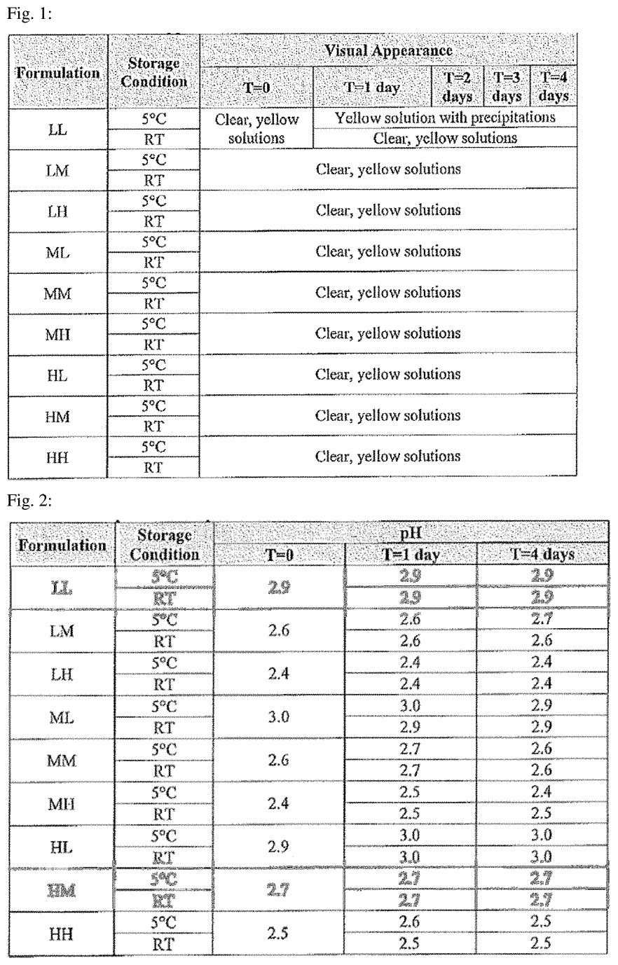

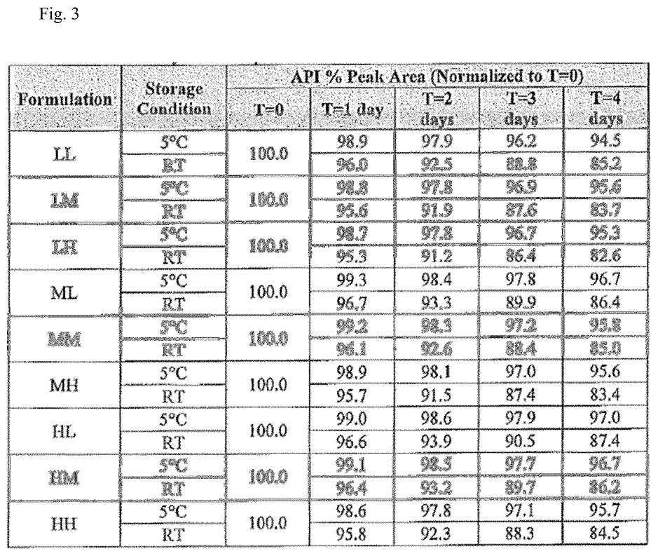

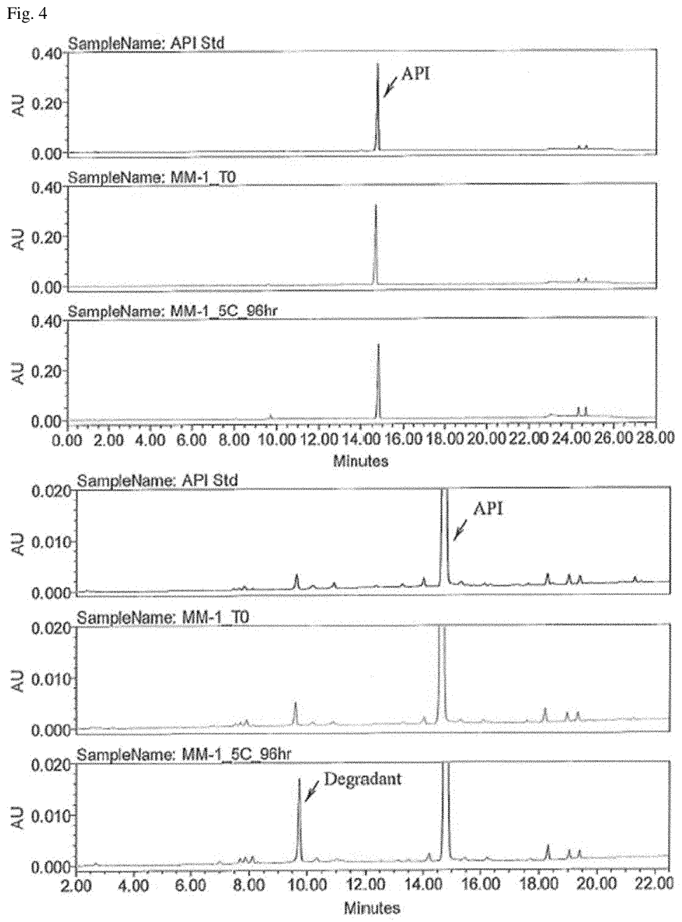

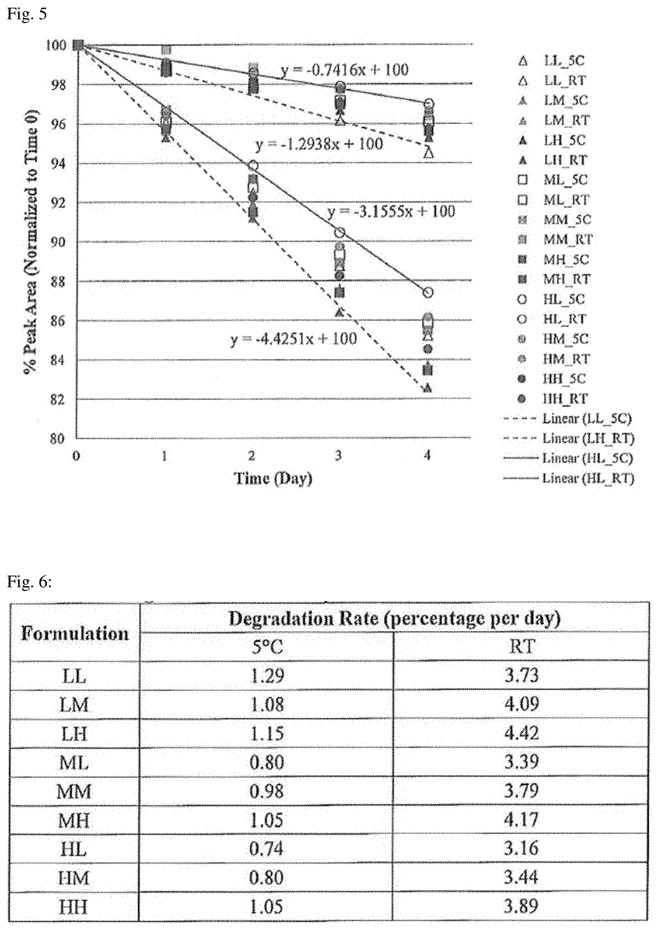

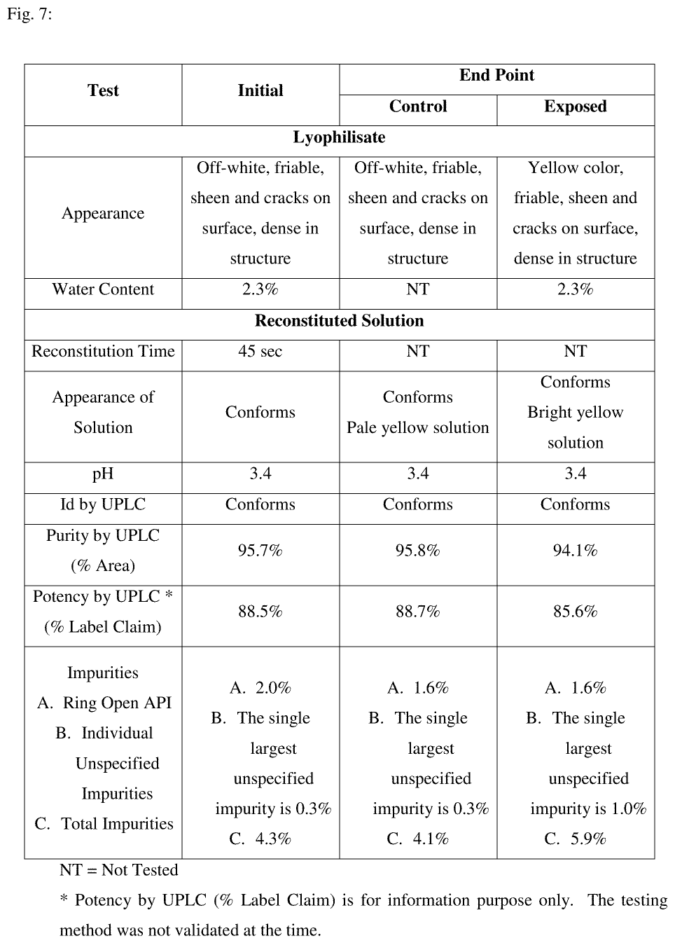

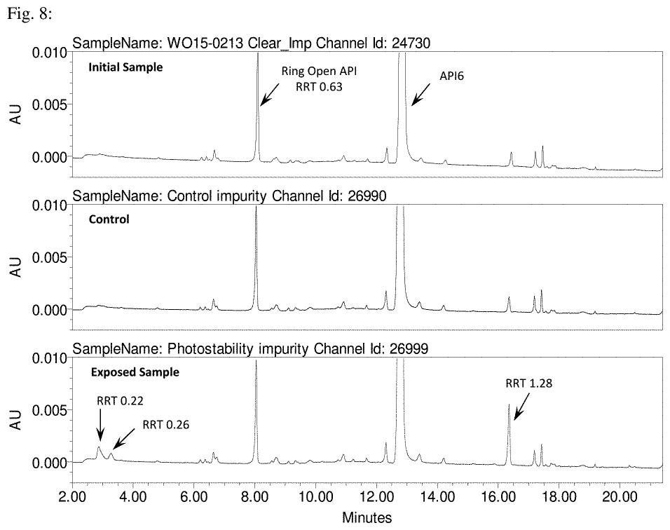

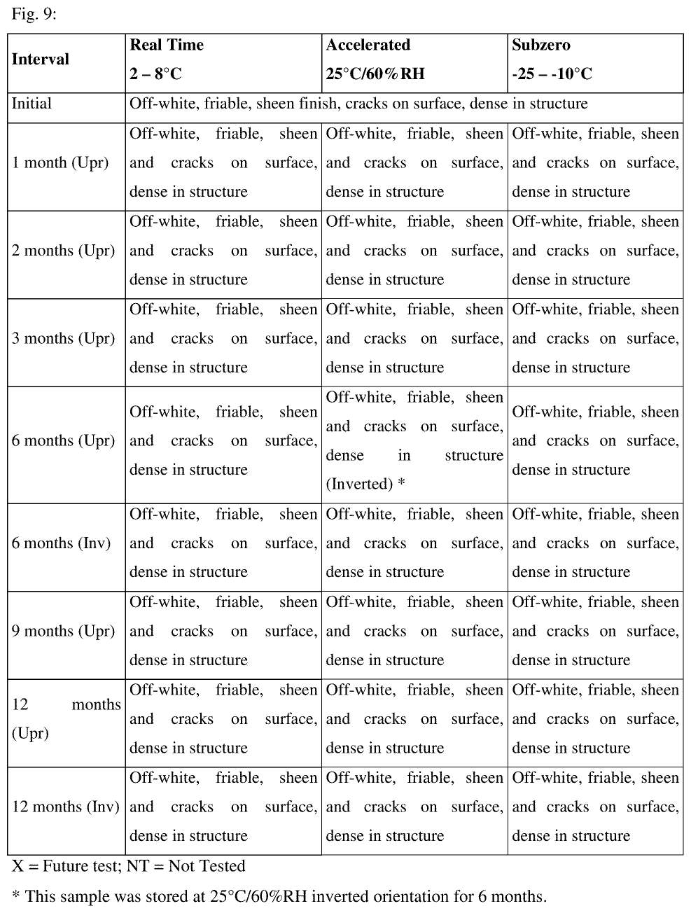

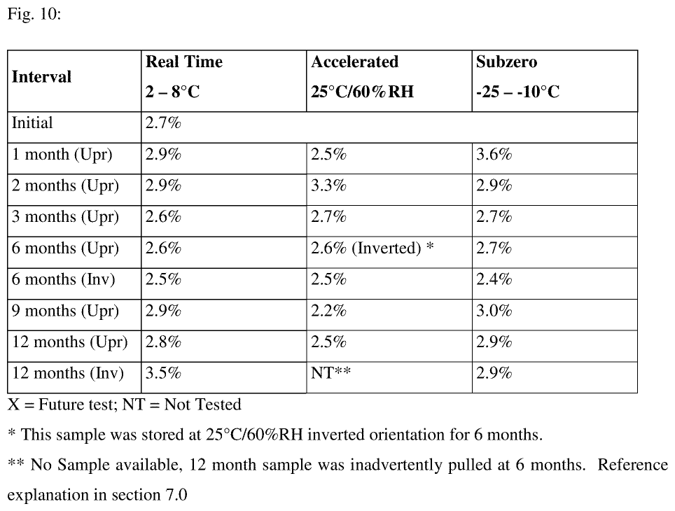

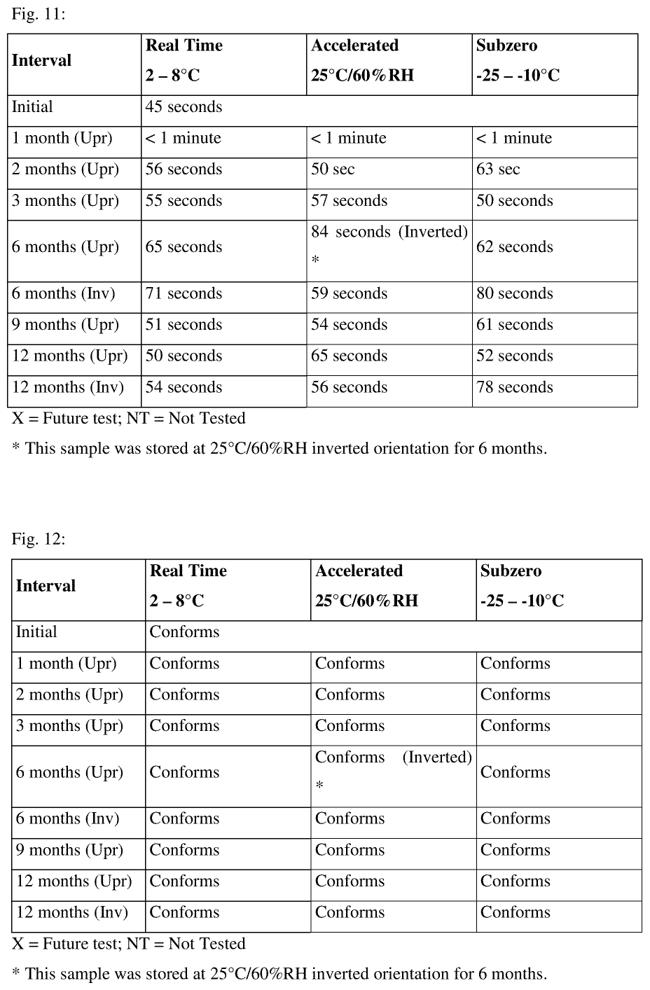

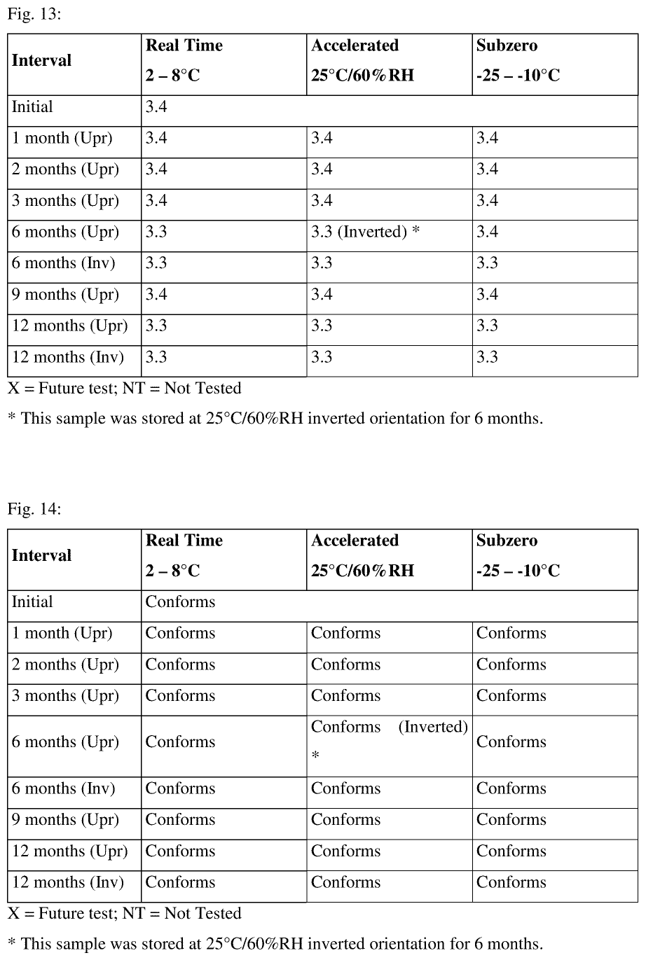

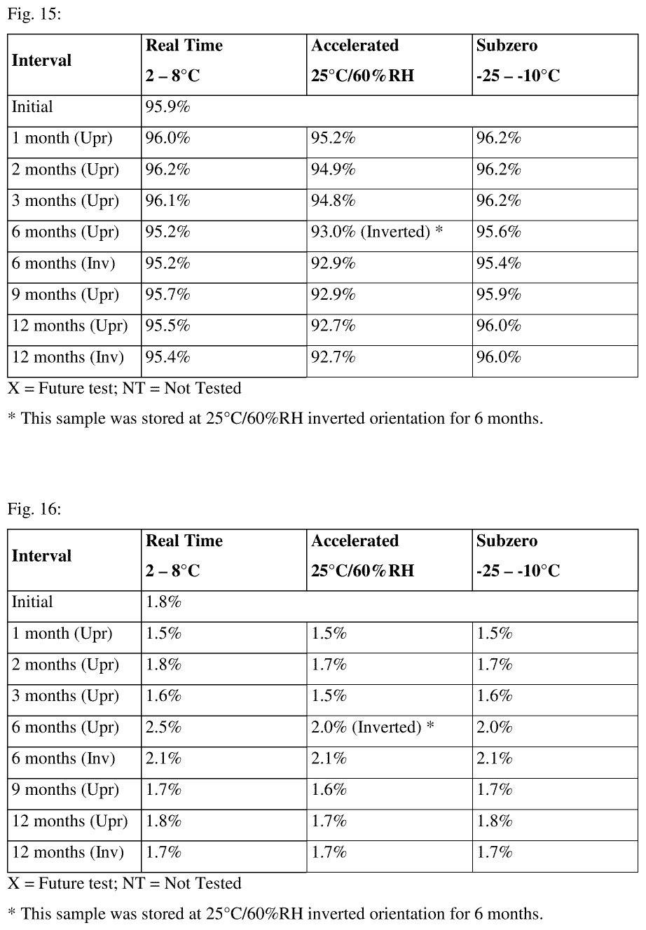

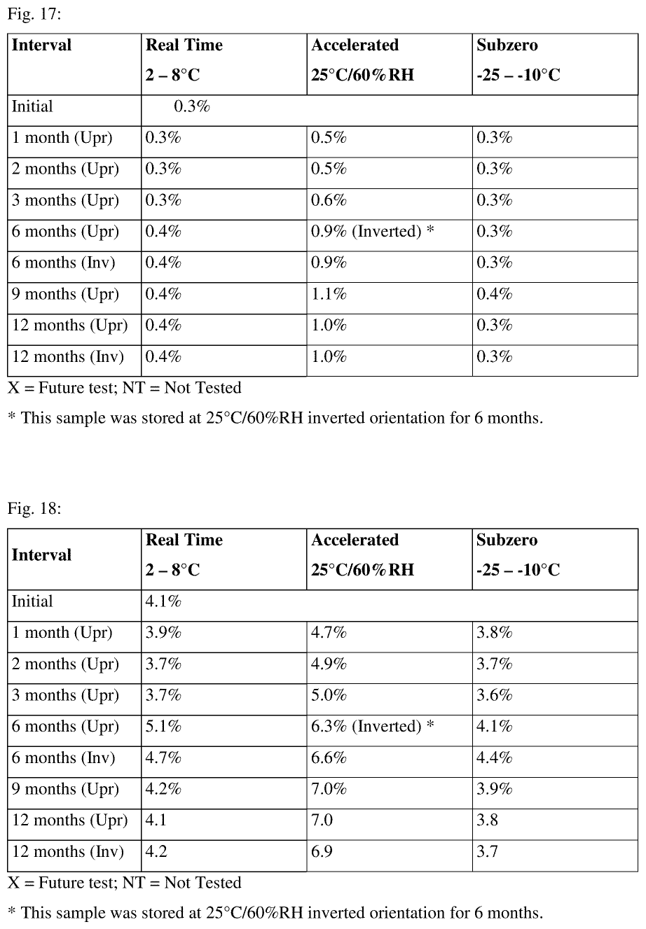

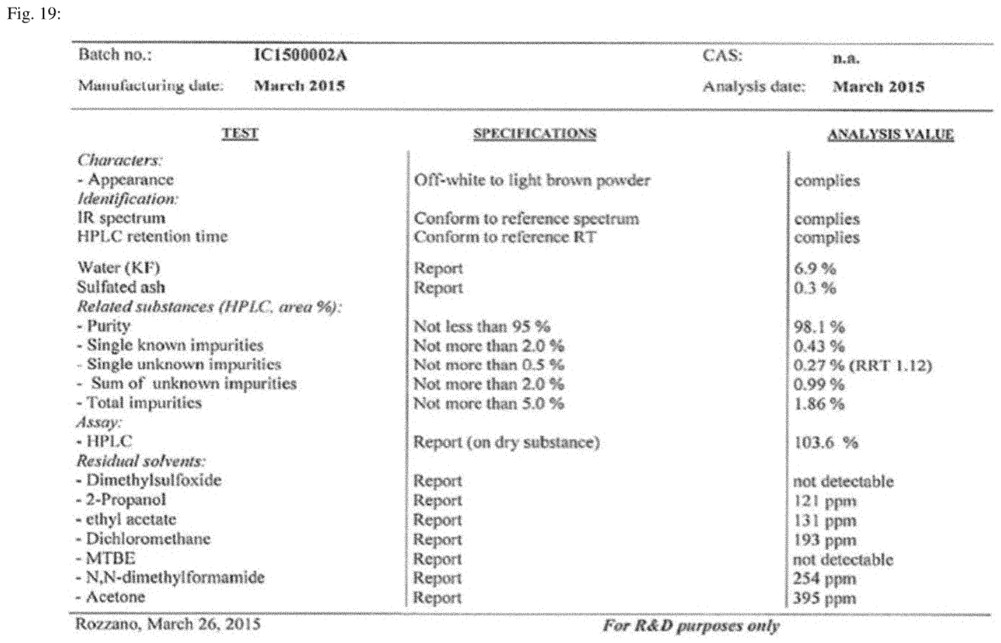

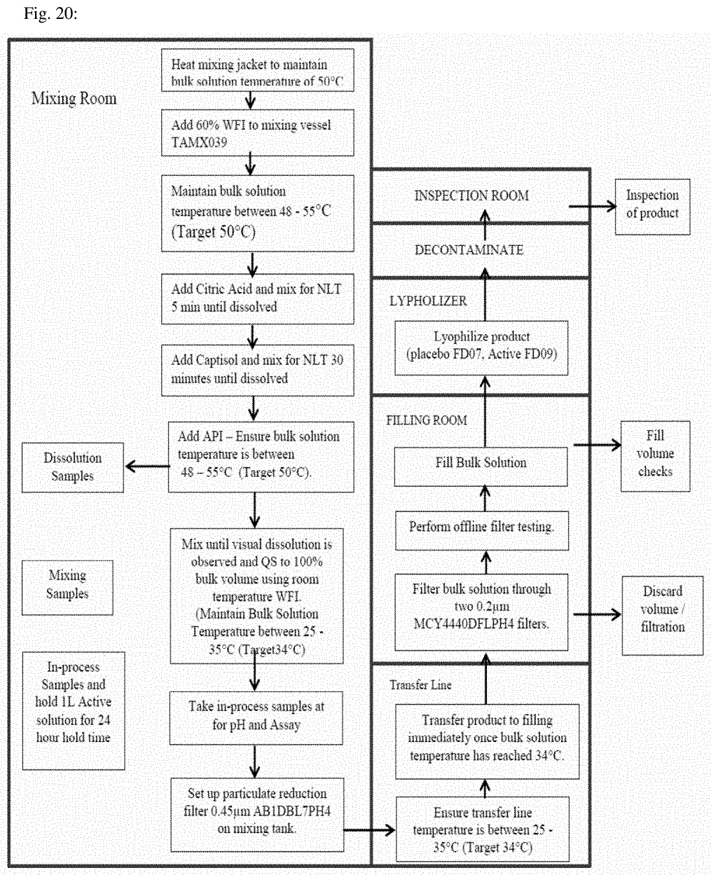

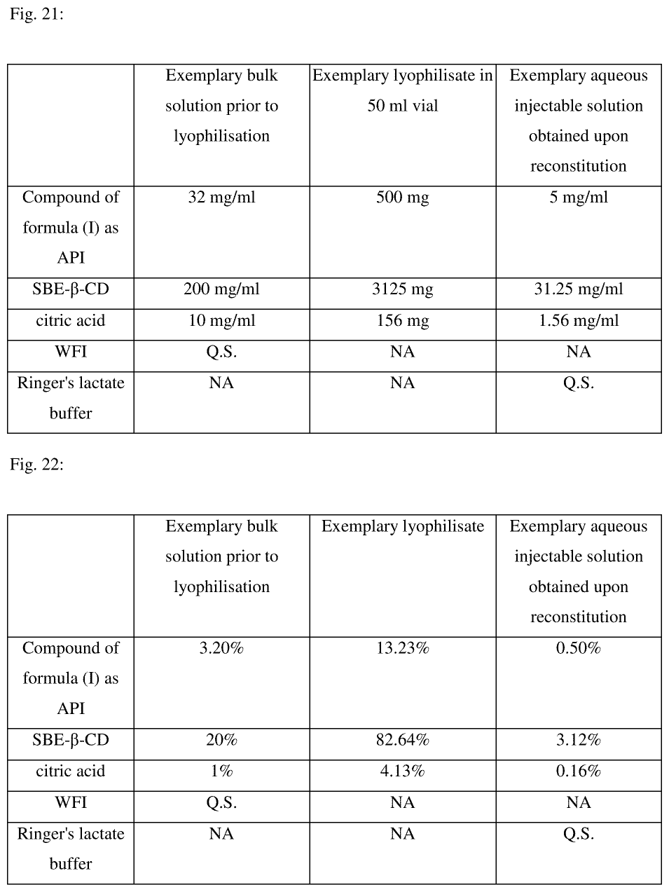

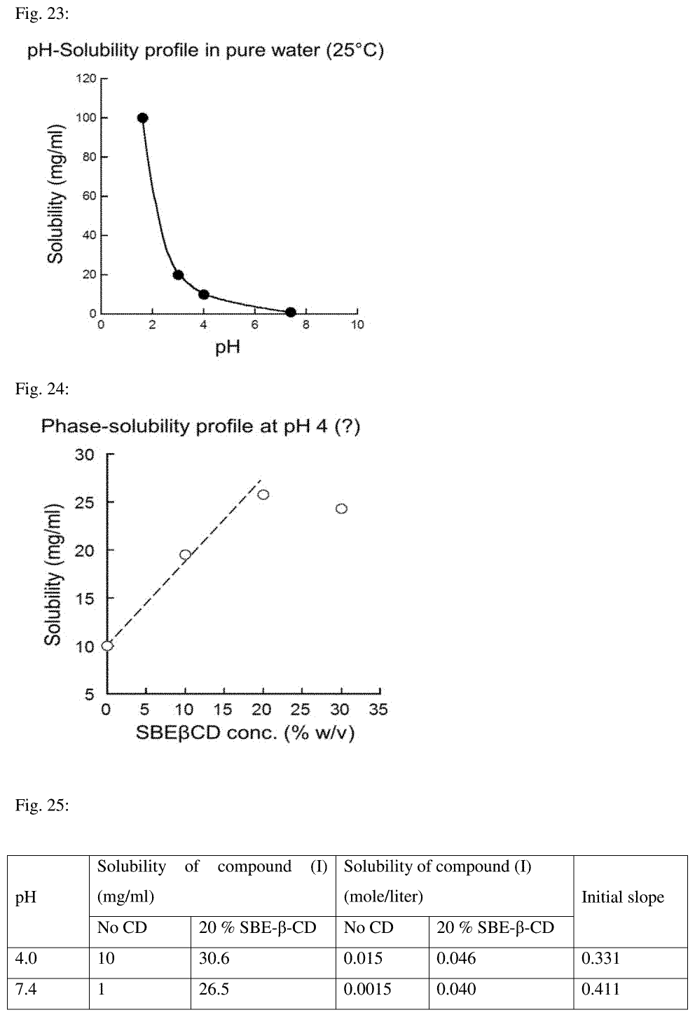

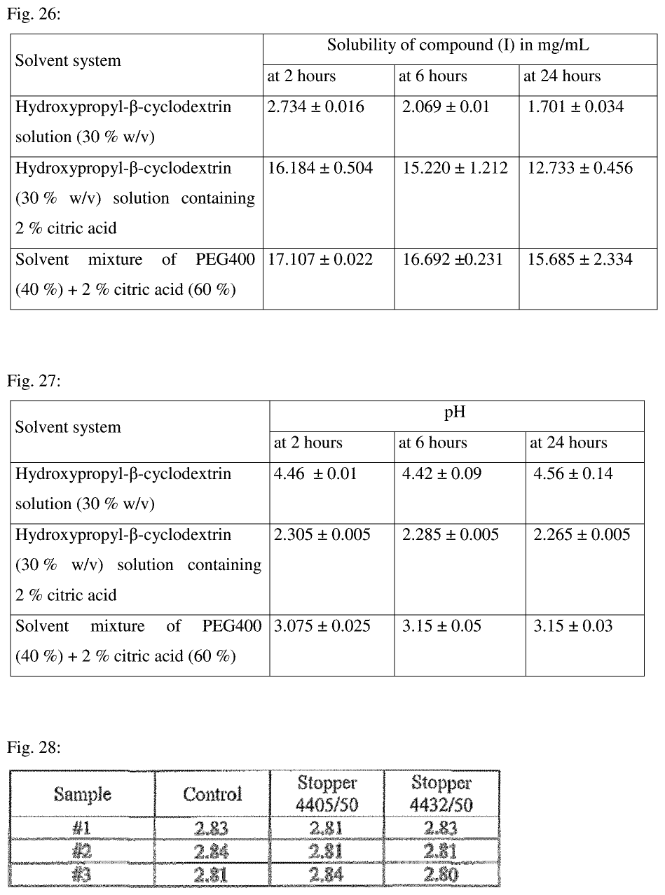

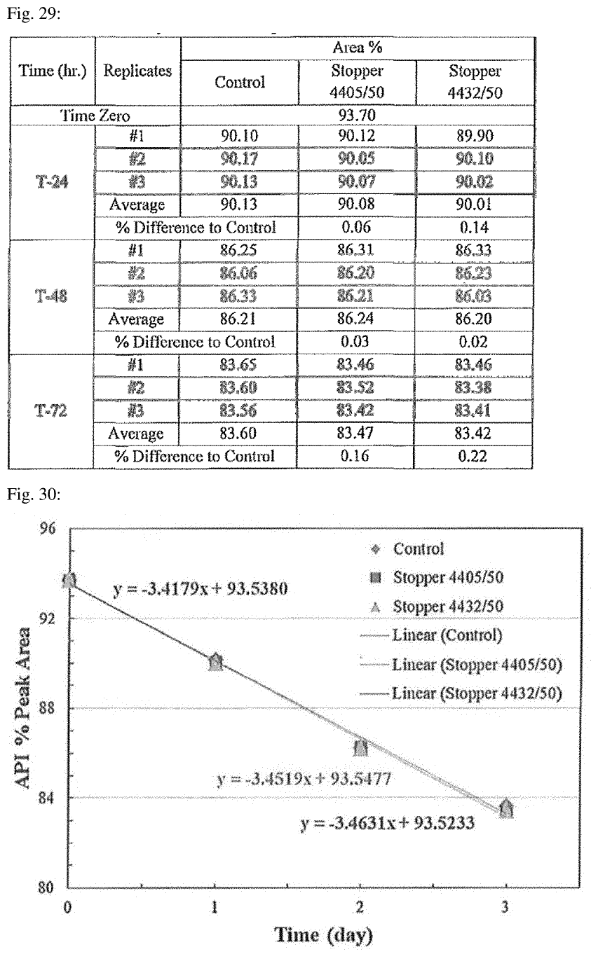

### DEFINITIONS

The “modified-cyclodextrin” or similar terms suitable for use herein refers to alpha-, beta-, gamma-cyclodextrins which have at least one modification to its structure when directly compared to cyclodextrin in general having the structure:

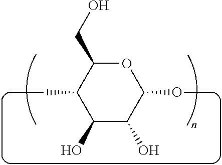

and when compared to the general structures of unmodified alpha-, beta-, gamma-cyclodextrins as depicted below:

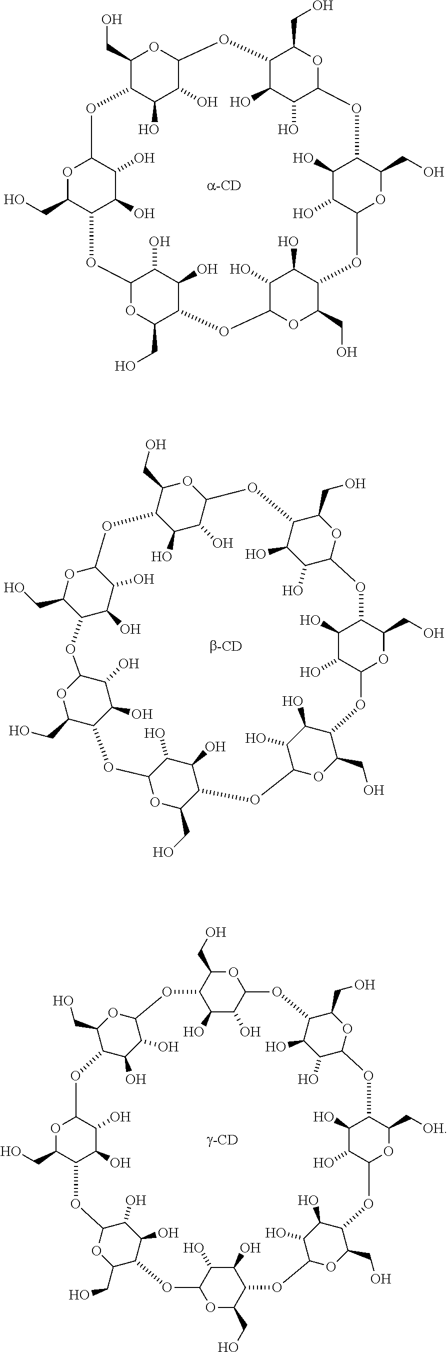

With this definition and the context of the invention, SBE-β-CD and HPB-β-CD are the preferred modified cyclodextrins, whereby SBE-β-CD is even more preferred.

Thus, a “modified cyclodextrin” is a cyclodextrin derivative compound in accordance with the invention and the below definitions apply:

| Cyclodextrin | Abbreviation | R | n |
|---|---|---|---|
| α-cyclodextrin | α-CD | H | 4 |
| β-cyclodextrin | β-CD | H | 5 |
| γ-cyclodextrin | γ-CD | H | 6 |
| Carboxymethyl-β-cyclodextrin | CM-β-CD | CH₂CO₂H or H | 5 |
| Carboxymethyl-ethyl- | CME-β-CD | CH₂CO₂H, CH₂CH₃ or H | 5 |
| β-cyclodextrin | | | |
| Diethyl-β-cyclodextrin | DE-β-CD | CH₂CH₃ or H | 5 |
| Dimethyl-β-cyclodextrin | DM-β-CD | CH₃ or H | 5 |
| Glucosyl-β-cyclodextrin | G₁-β-CD | Glucosyl or H | 5 |
| Hydroxybutenyl-β-cyclodextrin | HBU-β-CD | CH₂CH(CHCH₂)OH or H | 5 |
| Hydroxyethyl-β-cyclodextrin | HE-β-CD | CH₂CH₂₀H or H | 5 |
| Hydroxypropyl-β-cyclodextrin | HP-β-CD | CH₂CHOHCH₃ or H | 5 |
| Hydroxypropyl-γ-cyclodextrin | HP-γ-CD | CH₂CHOHCH₃ or H | 6 |
| Maltosyl-β-cyclodextrin | G₂-β-CD | Maltosyl or H | 5 |
| Methyl-β-cyclodextrin | M-β-CD | CH₃ or H | 5 |
| Random methyl-β-cyclodextrin | RM-β-CD | CH₃ or H | 5 |
| Sulfobutylether-β-cyclodextrin | SBE-β-CD | (CH₂)₄SO₃Na or H | 5 |

[symbol]

Derivatives may have differing degrees of substitution on the 2, 3, and 6 positions. [symbol]
indicates data missing or illegible when filed

With the context of the formulations and compositions of the invention, generally it applies that in aqueous solution the components are given in “w/v” units and in solid state (e.g. lyophilized state) the components are given in “w/w” units.

The expression “in-use stability” or similar expressions denote(s) a period of time during which the aqueous injectable formulations of the invention as medicinal products can be used in parenteral administration, preferably by i.v. injection, whilst retaining quality within an accepted specification once a container or bag containing said medicinal product is opened. This also includes aqueous injectable formulations of the invention as medicinal products which may be provided in multidose containers/bags which—by nature of their physical form and chemical composition—due to repeated opening and closing, may pose a risk to its content with regard to microbiological contamination, proliferation and/or physico-chemical degradation once the closure system has been breached. Testing of in-use stability may be followed according to the actual “Note for guidance on in-use stability testing of human medicinal products” published by the European Agency for the Evaluation of Medicinal Products.

The term “unit dosage form” is used herein to mean a single or multiple dose form containing a quantity of the active pharmaceutical ingredient (API) and the diluent or carrier, said quantity being such that one or more predetermined units are normally required for a single therapeutic administration. In the case of multiple dose forms, such as liquid-filled ampoules, said predetermined unit will be one fraction such as a half or quarter of the multiple dose form. It will be understood that the specific dose level for any patient will depend upon a variety of factors including the indication being treated, therapeutic agent employed, the activity of therapeutic agent, severity of the indication, patient health, age, sex, weight, diet, and pharmacological response, the specific dosage form employed and other such factors.

The expression “pharmaceutically acceptable” or similar expressions is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “patient” is taken to mean warm blooded animals such as mammals, for example, cats, dogs, mice, guinea pigs, horses, bovine cows, sheep, and humans.

The liquid formulation of the invention will comprise an effective amount of the API according to the aforementioned formulae (I) to (VII), whereby a compound (I) as API is preferred. By the term “effective amount” it is understood that a therapeutically effective amount of said API is contemplated. A therapeutically effective amount is the amount or quantity of API that is sufficient to elicit the required or desired antimicrobial response, or in other words, the amount that is sufficient to elicit an appreciable biological response when administered to a subject.

The expression “reconstitution time” determines the time by when all freeze dried product; i.e. a solid composition in accordance with the invention, is dissolved into clear solution.

The expressions “clear, clarity” or similar expressions with the context of the solutions disclosed herein refers to determination of clarity by visual inspection; however, other known methods for determining the clarity of a solution can be performed. Exemplary other methods include transmittance spectrophotometry at a wavelength of 800 nm. Using either method, solutions prepared according to the invention were determined to be at least visually clear. A clear liquid will generally contain no precipitate of the API.

The term “antimicrobial” or similar terms denote an agent or agent(s) that kills microorganisms or inhibits their growth; i.e. also denoted as “antimicrobials”. Antimicrobial medicines can be grouped according to the microorganisms they act primarily against. For instantce, “antibiotics” are used against bacteria and “antifungals” are used against fungi. Thus, in accordance with the instant invention, the term “antimicrobial” or similar terms can be interpreted as comprising “antibiotics” and “antifungals”, preferably “antimicrobial” can be interpreted as “antibacterial” with the context of the present invention.

“Antimicrobials” can also be classified according to their function. Agents that kill microbes are called microbicidal, while those that merely inhibit their growth are called biostatic. With the context of the invention, one of the main classes of antimicrobial agents are “antibiotics”, which generally destroy microorganisms within the body, preferably bacteria. The term “antibiotic” with the context of the invention describes both, those formulations derived from living organisms but it also applies to synthetic antimicrobials, such as the amidine substituted beta-lactam compounds of the invention. The term should be not construed to be restricted to antibacterials, rather its context should be broadened to include all antimicrobials. “Antibacterial agents” can be further subdivided into “bactericidal agents”, which kill bacteria, and “bacteriostatic agents”, which slow down or stall bacterial growth, and these mechanisms of action are also comprised by the meaning of “antimicrobials” or similar terms in accordance with the invention.

The expressions “zwitterionic, zwitterionic properties, and zwitterion” in the context of the present invention for the APIs of the compounds (I) to (VII) means that a compound molecule is a neutral molecule having a positive and a negative electrical charge at different locations within the same molecule. Accordingly, the API has a charge, which changes with pH when measured in an electric field. Thus, the compounds (I) to (VII) migrate in an electric field and the direction of migration depends upon the net charge possessed by the molecules. The net charge is influenced by the pH value.

The terms “dissolution, dissolution properties” denote the process or the characteristic by which a solid, liquid or gas forms a solution in a solvent. For the dissolution of solids, the process of dissolution can be explained as the breakdown of the crystal lattice into individual ions, atoms or molecules and their transport into the solvent. Overall the free energy must be negative for net dissolution to occur.

By contrast, “solubility” is the property of a solid, liquid, or gaseous chemical substance called solute to dissolve in a solid, liquid, or gaseous solvent to form a homogeneous solution of the solute in the solvent. The solubility of a substance fundamentally depends on the used solvent as well as on temperature and pressure. The extent of the solubility of a substance in a specific solvent is measured as the saturation concentration, where adding more solute does not increase the concentration of the solution. Solubility is not to be confused with the ability to dissolve or liquefy a substance, because the solution might occur not only because of dissolution but also because of a chemical reaction. Solubility does neither depend on particle size or other kinetic factors; given enough time, even large particles will eventually dissolve.

The term “bioavailability” denotes in general a subcategory of absorption and is the fraction of an administered dose of an API that reaches the systemic circulation, one of the principal pharmacokinetic properties of drugs. By definition, when a medication is administered intravenously, its bioavailability is 100%. However, when a medication is administered via other routes (such as orally), its bioavailability generally decreases (due to incomplete absorption and first-pass metabolism) or may vary from individual to individual.

Bioavailability is one of the essential tools in pharmacokinetics, as bioavailability must be considered when calculating dosages for non-intravenous routes of administration.

### ABBREVIATIONS

API active pharmaceutical ingredient; i.e. with the context of the invention, a compound of the formulae (I)-(VII) and the salts thereof, the solvates thereof and the solvates of the salts thereof
CA citric acid
HP-β-CD hydroxypropyl-β-cyclodextrin
i.v. intravenous/intravenously
SBE-β-CD sulfobutyl ether-β-cyclodextrin
q.s. quantum satis (e.g. quantum satis aqueous solution)
WFI water for injection
RH relative humidity
PVDF Polyvinylidene difluoride

1. A formulation comprising a compound selected from a group of compounds consisting of the formulae (I) to (VII): 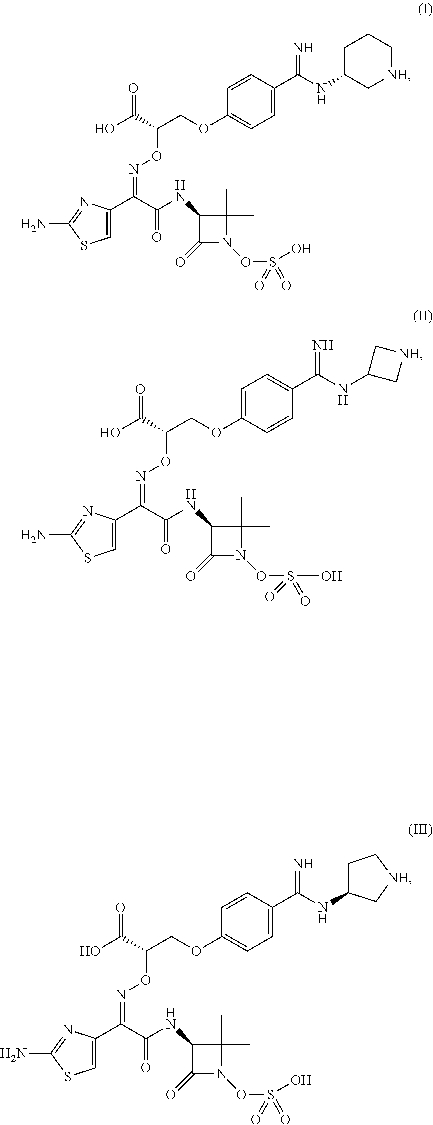 [SMILES: CC1(C)[C@H](NC(=O)/C(=N\O[C@@H](COc2ccc(C(=N)N[C@@H]3CCCNC3)cc2)C(=O)O)c2csc(N)n2)C(=O)N1OS(=O)(=O)O ; CC1(C)[C@H](NC(=O)/C(=N\O[C@@H](COc2ccc(C(=N)NC3CNC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCNC3)cc2)C(=O)O)c2csc(N)n2)C(=O)N1OS(=O)(=O)O] 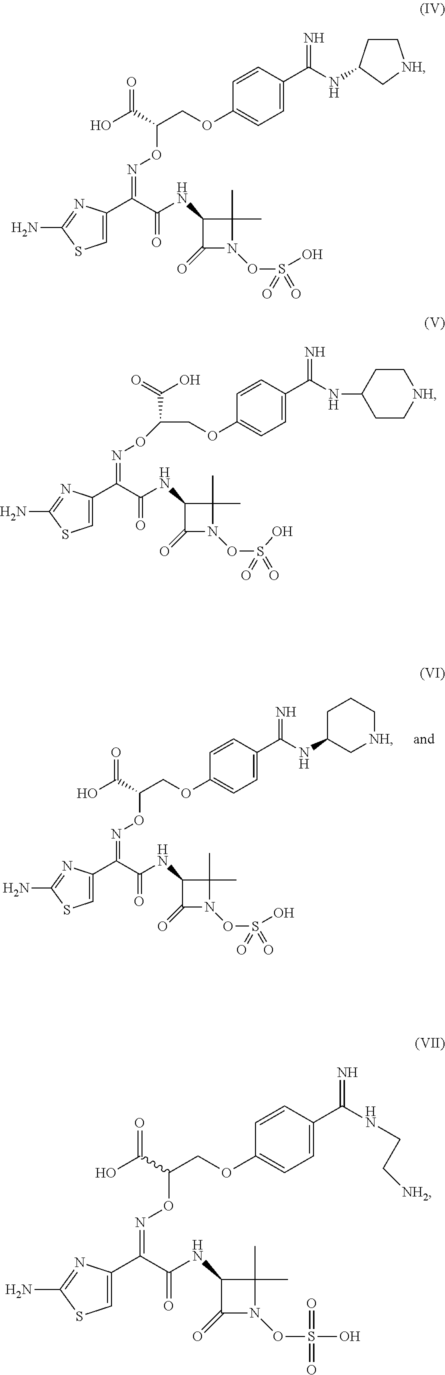 [SMILES: CC1(C)[C@H](NC(=O)/C(=N\O[C@@H](COc2ccc(C(=N)N[C@@H]3CCNC3)cc2)C(=O)O)c2csc(N)n2)C(=O)N1OS(=O)(=O)O ; CC1(C)[C@H](NC(=O)/C(=N\O[C@@H](COc2ccc(C(=N)NC3CCNCC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCCNC3)cc2)C(=O)O)c2csc(N)n2)C(=O)N1OS(=O)(=O)O ; "and" ; CC1(C)[C@H](NC(=O)/C(=N\OC(COc2ccc(C(=N)NCCN)cc2)C(=O)O)c2csc(N)n2)C(=O)N1OS(=O)(=O)O]
or the salts thereof, the solvates thereof or the solvates of the salts thereof, and further comprising
a) an organic acid selected from the group comprising citric acid, tartaric acid, malic acid, maleic acid, methanesulphonic acid, ascorbic acid, adipic acid, aspartatic acid, benzenesulfonic acid, glucoheptonic acid, D-gluconic acid, L-glutamic acid, lactic acid, L-Lysine, saccharin; and/or
b) an inorganic acid selected from the group comprising hydrochloric acid, sulfuric acid, phosphoric acid, and nitric acid; and
c) a modified cyclodextrin in aqueous solution,
wherein
i) said compound of the formulae (I)-(VII) has a concentration in the range of 1-5% w/v, with the proviso that at least an organic acid according to a) is used, and wherein said organic acid has a concentration in the range of 0.25-4% w/v, or
ii) wherein said compound of the formulae (I)-(VII) has a concentration in the range of 1-15% w/v, with the proviso that only an inorganic acid according to b) is used, and
wherein either for i) or ii) said inorganic acid has a concentration in the range of 0.25-6% w/v, and
wherein either for i) or ii) said modified cyclodextrin has a concentration in the range of 10-40% w/v in said aqueous solution, and
wherein either for i) or ii) said formulation has a pH in the range of 1.25 to 2.8.

2. The formulation according to claim 1, wherein said modified cyclodextrin in aqueous solution is selected from a group comprising α-cyclodextrin, β-cyclodextrin, γ-cyclodextrin or a modified derivative thereof.

3. The formulation according to claim 2, wherein said β-cyclodextrin or a modified derivative thereof is selected from a group comprising hydroxypropyl-β-cyclodextrin and sulfobutyl ether-β-cyclodextrin.

4. The formulation according to claim 1, wherein said organic acid is selected from a group comprising citric acid, tartaric acid, malic acid, maleic acid, methanesulphonic acid, ascorbic acid, L-Lysine, and saccharin.

5. The formulation according to claim 1, wherein said inorganic acid is selected from a group comprising hydrochloric acid, sulfuric acid, and phosphoric acid.

6. The formulation according to claim 1, wherein said modified cyclodextrin is sulfobutyl ether-beta-cyclodextrin (captisol) and said organic acid is citric acid.

7. A solid composition, wherein said solid composition is comprising at least one compound according to the formulae (I)-(VII) as defined in claim 1, and at least one modified cyclodextrin selected from the group consisting of α-cyclodextrin, β-cyclodextrin, γ-cyclodextrin or a modified derivative thereof with a concentration of up to 95% w/w, and at least one organic acid with a concentration of up to 20% w/w, selected from the group consisting of citric acid, tartaric acid, malic acid, maleic acid, methanesulphonic acid, ascorbic acid, L-lysine, and saccharin and/or at least one inorganic acid with a concentration of up to 25% w/w selected from the group consisting of hydrochloric acid, sulfuric acid, and phosphoric acid.

8. A solid composition according to claim 7, wherein said modified cyclodextrin is sulfobutyl ether-beta-cyclodextrin (captisol) and said organic acid is citric acid.

9. A solid composition according to claim 7, wherein said solid composition is further characterized by a stability of said compound according to the formulae (I)-(VII) over 12 months at 25° C./60% relative humidity, or 2-8° C. ambient temperature, or at −20° C. ambient temperature storing condition.

10. A solid composition according to claim 7 obtained by lyophilization.

11. A pharmaceutical formulation obtainable from the solid composition as defined in claim 7.

12. A pharmaceutical formulation according to claim 11, wherein said formulation comprises a compound according to any of formulae (I) to (VII) at 6-15%, preferably at 13.2%;
Captisol at 60-95%, preferably at 82%, and citric acid at 2-10%, preferably at 4.1%.

13. A pharmaceutical formulation according to claim 11, wherein said formulation comprises a compound according to any of formulae (I) to (VII) at 13.2%;
Captisol at 82%, and citric acid at 4.1%.

14. A pharmaceutical formulation according to claim 13, wherein said formulation is obtainable from a solid composition upon reconstitution in a suitable aqueous medium of a lyophilized formulation of a compound of any of formulae (I) to (VII).

15. A pharmaceutical formulation according to claim 14, wherein said pharmaceutical formulation is further characterized by an in-use stability of said compound according to the formulae (I)-(VII) in the reconstituted aqueous solution for over 24 hours at room temperature.

16. An aqueous injectable formulation comprising a compound of the formulae (I)-(VII) as defined in claim 1, a modified cyclodextrin selected from the group consisting of α-cyclodextrin, β-cyclodextrin, γ-cyclodextrin or a modified derivative thereof, an organic and/or inorganic acid selected from the group consisting of hydrochloric acid, sulfuric acid, and phosphoric acid, citric acid, tartaric acid, malic acid, maleic acid, methanesulphonic acid, ascorbic sold, L-Lysine, and saccharin and water, wherein said aqueous injectable formulation is having a pH within the range as of from 4.0 to 4.5.

17. A process for the preparation of a formulation as defined in claim 1, said process comprising the steps of:
i) providing a means for mixing, preferably a mixing tank,
ii) maintaining the bulk solution temperature at approx. 50° C. by heating means, preferably by using a heat mixing jacket,
iii) adding approx. 60% w/v water for injection, preferably adding hot 60% w/v water for injection at approx. 60° C.,
iv) maintaining bulk solution temperature at a range of 48-55° C., preferably 49-52° C., most preferred at 50° C., whereby 50° C. is the target temperature,
v) adding an organic and/or an inorganic acid in accordance with the invention and mix the solution, preferably mix at least for 3 minutes, more preferred at least for 4 minutes, most preferred at least for 5 minutes until dissolved,
vi) adding a modified cyclodextrin in accordance with the invention and mix the solution, preferably mix at least for 20 minutes, more preferred at least for 25 minutes, most preferred at least for 30 minutes until dissolved,
vii) adding a compound of the formulae (I) to (VII) as API in accordance with the invention and ensure that bulk solution temperature is at a range of 48-55° C., preferably 49-52° C., most preferred at 50° C., whereby 50° C. is the target temperature,
viii) mixing the solution obtained under step vii) until visual dissolution is observed, and fill up to 100% bulk volume using water for injection at room temperature, thereby maintaining bulk solution at 25-35° C., preferably at 29-35° C., most preferred at 34-35° C., whereby 34-35° C. is the target temperature,
ix) optionally take in-process sample(s) to monitor pH or for using other assays
x) setting up a particulate reduction filter, preferably a 0.45 μm particulate reduction filter, on mixing means, preferably on mixing tank
xi) ensuring transfer line temperature is at 25-35° C., preferably at 29-35° C., most preferred at 34-35° C., whereby 34-35° C. is the target temperature,
xii) transferring the product of step xi) immediately to filling room, as soon as bulk solution reached a temperature at 34-35° C. as target temperature,
xiii) filtering bulk solution of step xii) through a suitable filter, preferably a 0.2 μm filter, more preferably through two 0.2 μm filter, whereby even more preferably said filter is a Polyvinylidene difluoride membrane (PVDF)
xiv) optionally perform offline filter testing
xv) filling bulk solution.

18. A process for the preparation of a solid composition as defined in claim 7, said process comprising the steps of:
i) providing a means for mixing, preferably a mixing tank,
ii) maintaining the bulk solution temperature at approx 50° C. by heating means, preferably by using a heat mixing jacket,
iii) adding approx. 60% w/v water for injection, preferably adding hot 60% w/v water for injection at approx. 60° C.,
iv) maintaining bulk solution temperature at a range of 48-55° C., preferably 49-52° C., most preferred at 50° C., whereby 50° C. is the target temperature,
v) adding an organic and/or an inorganic acid in accordance with the invention and mix the solution, preferably mix at least for 3 minutes, more preferred at least for 4 minutes, most preferred at least for 5 minutes until dissolved,
vi) adding a modified cyclodextrin in accordance with the invention and mix the solution, preferably mix at least for 20 minutes, more preferred at least for 25 minutes, most preferred at least for 30 minutes until dissolved,
vii) adding a compound of the formulae (I) to (VII) as API in accordance with the invention and ensure that bulk solution temperature is at range of 48-55° C., preferably 49-52° C., most preferred at 50° C., whereby 50° C. is the target temperature,
viii) mixing the solution obtained under step vii) until visual dissolution is observed, and fill up to 100% bulk volume using water for injection at room temperature, thereby maintaining bulk solution at 25-35° C., preferably at 29-35° C., most preferred at 34-35° C., whereby 34-35° C. is the target temperature,
ix) optionally take in-process sample(s) to monitor pH or for using other assays
x) setting up a particulate reduction filter, preferably a 0.45 μm particulate reduction filter, on mixing means, preferably on mixing tank
xi) ensuring transfer line temperature is at 25-35° C., preferably at 29-35° C., most preferred at 34-35° C., whereby 34-35° C. is the target temperature,
xii) transferring the product of step xi) immediately to filling room, as soon as bulk solution reached a temperature at 34-35° C. as target temperature,
xiii) filtering bulk solution of step xii) through a suitable filter, preferably a 0.2 μm filter, more preferably through two 0.2 μm filter, whereby even more preferably said filter is a Polyvinylidene difluoride membrane (PVDF)
xiv) optionally perform offline filter testing
xv) filling bulk solution,
xvi) lyophilizing the product obtained under step xv)
xvii) and optionally decontaminating the lyophilized product obtained under step xvi.

19. A process for the preparation of an aqueous injectable solution as defined in claim 18, comprising the steps of:
xviii) reconstituting the lyophilisate obtained in step xvi) and optionally step xvii) of claim 18 with a suitable medium comprising water for injection, NaCl solution, dextrose solution, and Ringer's lactate solution, followed by
xix) adding phosphate buffer/saline mixture solution for pH adjustment, so to obtain a final aqueous injectable solution for use in parenteral administration with a pH value of 4.0 to 4.5 and an osmolality of 290 to 450 mOSM/kg.
